(19) United States
(12) Patent Application Publication
(10) Pub. No.: US 2019/0262400 A1
(43) Pub. Date: Aug. 29, 2019

(54) ARTIFICIAL ANTIGEN PRESENTING CELLS FOR EXPANDING IMMUNE CELLS FOR IMMUNOTHERAPY

(71) Applicant: H. LEE MOFFITT CANCER CENTER AND RESEARCH INSTITURE, INC., Tampa, FL (US)

(72) Inventor: Marco Davila, Tampa, FL (US)

(21) Appl. No.: 16/346,226

(22) PCT Filed: Oct. 31, 2017
(86) PCT No.: PCT/US2017/059253

§ 371 Date: Apr. 30, 2019

Related U.S. Application Data
(60) Provisional application No. 62/415,053, filed on Oct. 31, 2016.

(51) Int. Cl. A61K 35/17; A61P 35/00; C07K 14/725; C07K 16/28; C07K 14/705; C07K 14/54; C07K 14/55; C12N 5/0783

(52) U.S. Cl. CPC A61K 35/17; A61P 35/00; C07K 14/7051; C07K 16/2827; C07K 14/70578; A61K 2035/124; C07K 14/55; C07K 14/5418; C07K 14/5434; C07K 14/5428; C12N 5/0636; C07K 14/5443

(57) ABSTRACT

Disclosed herein are methods of expanding immune cells for immunotherapy using artificial antigen presenting cells (aAPCs) having on their surface antibodies or ligands that bind molecules of both the T cell activation pathway and T cell costimulation pathway. The disclosed aAPCs can also secrete antibodies that bind molecules of the T cell inhibitory pathway. For example, anti-CD3 scFv on the surface of the aAPCs can bind and activate T cells, while anti-CD28 scFv and 4-1BBL on the surface of the aAPCs can provide dual co-stimulation for the T cells resulting in decreased levels of the markers CD25, TIM3, LAG3, and PD1. For example, blocking PD1/PDL1 ligation can limit suppression that is mediated by the tumor microenvironment. This is a less costly and more efficient alternative to peripheral blood mononuclear cells (PBMCs) and cytokine treatments that result in better quality T cell for adoptive transfer back into patients.

### CROSS-REFERENCE TO RELATED APPLICATIONS

This application claims benefit of U.S. Provisional Application No. 62/415,053, filed Oct. 31, 2016, incorporated herein by reference in its entirety.

### BACKGROUND

Adoptive cell therapy (ACT) using tumor infiltrating lymphocytes (TIL) can lead to positive, objective, and durable responses in cancer patients. However, this therapy can involve sophisticated cell processing techniques and equipment. These procedures have introduced technical, regulatory, and logistic challenges to the successful use of TIL as a biological therapy. Accordingly, there is a need in the art for improved methods for growing TIL for use in adoptive cell therapy.

### SUMMARY

Disclosed herein are methods of expanding immune cells for immunotherapy using artificial antigen presenting cells (aAPCs) having on their surface antibodies (including, but not limited to antibody fragments, such as, for example, F(ab′)2, Fab′, Fab, and/or scFv) or ligands that bind molecules of both the T cell activation pathway and T cell co-stimulation pathway. The disclosed aAPCs can also secrete antibodies that bind molecules of the T cell inhibitory pathway. For example, anti-CD3 scFv on the surface of the aAPCs can bind and activate T cells, while anti-CD28 scFv and 4-1BBL on the surface of the aAPCs can provide dual co-stimulation for the T cells resulting in decreased levels of the markers CD25, TIM3, LAG3, and PD1. This is a less costly and more efficient alternative to peripheral blood mononuclear cells (PBMCs) and cytokine treatments that result in better quality T cell for adoptive transfer back into patients.

In some embodiments, the disclosed aAPCs secrete an antibody (e.g. anti-PD1 or PDL1) that interferes with suppression of T cells, e.g. by ligation of PD1 with PDL1. This suppression is a normal physiologic immune response meant to prevent over-activation of T cells. However, cancer cells have co-opted this suppression pathway as a means to evade immune recognition and tumor killing. This system is a less costly, more efficient and more rapid alternative to peripheral blood mononuclear cells (PBMCs) and cytokine treatments. The system is less costly because a renewable resource replaces the need for cytokines, antibodies for activation, and PBMC feeders. The faster production time is also clinically meaningful considering that patients have to wait a few months for production of their cells, which can be a difficult task for patients with metastatic cancer. Also, extended culture often produce terminally differentiated T cells that have limited function and persistence when adoptively transferred back into patients. The shorter culture time therefore may allow us to infuse a T cell product that is more physiologic and tumor-reactive.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates the prior art method for expansion of tumor infiltrating lymphocytes (TILs) for infusion into a patient with a melanoma.

FIG. 2 depicts an example aAPC that expresses molecules to activate and co-stimulate a T cell and prevent T cell suppression.

FIG. 3 illustrates a method for expansion of marrow-infiltrating lymphocytes (MILs) for infusion into a patient with an acute myelogenous leukemia (AML) according to one disclosed embodiment.

FIG. 4 illustrates methods for expansion of tumor infiltrating lymphocytes (TILs) for infusion into a patient with a melanoma according to one disclosed embodiment that replaces A) peripheral blood mononuclear cells (PBMCs) with artificial antigen presenting cells (aAPCs) or another embodiment B) that cultures digested tumor fragments with aAPC.

FIGS. 5A to 5D show fold change of live (FIGS. 5A and 5C) and total (FIGS. 5B and 45) bone marrow T (BM-T) cells after stimulation with Dynabeads® or aAPCs for 7 days (FIGS. 5A and 5B) and 14 days (FIGS. 5C and 5D).

FIG. 6A to 6E show gating strategy for counting live CD3+ T cells stimulation with Dynabeads® or aAPCs.

FIGS. 7A to 7C show gating strategy for counting CD3+/CD4+ and CD3+/CD8+ cells after stimulation with Dynabeads® or aAPCs.

FIGS. 8A to 8C show gating strategy for counting CD4+/TIM3+ cells after stimulation with Dynabeads® or aAPCs.

FIGS. 9A to 9C show gating strategy for counting CD8+/TIM3+ cells after stimulation with Dynabeads® or aAPCs.

FIGS. 10A to 10C show gating strategy for counting CD4+/LAG3+ cells after stimulation with Dynabeads® or aAPCs.

FIGS. 11A to 11C show gating strategy for counting CD8+/LAG3+ cells after stimulation with Dynabeads® or aAPCs.

FIGS. 12A to 12C show gating strategy for counting CD4+/PD1+ cells after stimulation with Dynabeads® or aAPCs.

FIGS. 13A to 13C show gating strategy for counting CD8+/PD1+ cells after stimulation with Dynabeads® or aAPCs.

FIGS. 14A to 14C show gating strategy for counting CD4+/CD25+ cells after stimulation with Dynabeads® or aAPCs.

FIGS. 15A to 15C show gating strategy for counting CD8+/CD25+ cells after stimulation with Dynabeads® or aAPCs. All groups decreased CD25 on CD4 cells, but APCs containing 4-1BBL decreased the most.

FIGS. 16A to 16C show gating strategy for counting CD4+/CD69+ cells after stimulation with Dynabeads® or aAPCs.

FIGS. 17A to 17C show gating strategy for counting CD8+/CD69+ cells after stimulation with Dynabeads® or aAPCs.

FIGS. 18A to 18C show gating strategy for counting CD4+/CD137+ cells after stimulation with Dynabeads® or aAPCs.

FIGS. 19A to 19C show gating strategy for counting CD8+/CD137+ cells after stimulation with Dynabeads® or aAPCs.

FIGS. 20A to 20C show gating strategy for counting CD4+/CD28+ cells after stimulation with Dynabeads® or aAPCs.

FIGS. 21A to 21C show gating strategy for counting CD8+/CD28+ cells after stimulation with Dynabeads® or aAPCs.

FIG. 22A shows four normal donor T cells were expanded with K562 empty as negative control, human CD3CD28 beads, K562hCD3CD28scFv APCs for 14 days; the other four normal donor T cells were also expanded with human CD3CD28CD137 beads, K562hCD3scFvCD28scFvCD137L APCs and negative control K562 cells. The flow phenotype experiments were performed on day0, day7 and day14. Compared to K562 empty control, every expansion group significantly increased CD3 health donor T cells on day7 and day14, the triple APCs increased 260 to 400 fold on day14 and achieved the best, the double beads achieved the second best from 180 to 250 fold on day14.

FIG. 22B shows CD8 increased in each expansion group when compared with K562 empty control on both day7 and day14, the triple APCs expansion group increased the most from 430 to 720 fold on day14, the double APCs and triple beads group achieved the second best from 140 to 330 fold on day14.

FIG. 22C shows CD4 fold increase showed the exact same pattern as CD8 and CD3 while compared to K562 control, the triple APCs and the double beads expansion group had very seminar increase (mean=164.24 and 136.68) on day14, they both significantly increased CD4 compared to double APCs and triple Beads group on day14.

FIG. 23A shows CD8 central memory T cells in each expansion group increased compared with K562 empty control group on both day7 and day14, and triple APCs increased CD8 TCM from 1000 to 24,000 fold (mean=8654) on day14 and continue to increase TCM from day7 to day14.

FIG. 23B shows CD8 TEM increased in double beads and double APCs while compared with K562 empty group from 20-500 fold on both day7 and day14, triple beads did not increase TEM on both time points since the baseline from one of the donor T cells was quite high. TEM of triple APCs did not increase on day7, but increased dramatically from 700 to 13,000 fold when compared with control on day14; triple APCs group increased the most while compared with double beads and double APCs on day14; and is the only group that continue increase from day7 to day14.

FIG. 23C shows CD8 TEFF increased in each expansion group on both day7 and day14 except triple APCs did not increase on day7; and all the expansion groups continue increasing TEFF from day7 to day14 but not K562 empty group. Both triple APCs (450-2,100 fold) and double beads (540-2,400 fold) had very good CD8 TEFF increase, the average fold is 910 for triple APCs and 1200 for double beads.

FIG. 24A shows that each group significantly increased CD3 by the time of expansion compare with control K562 empty except for K562hCD3CD28 double APCs on Day14, group K562hCD3CD28CD137L triple APCs increased the most and continued to increase the most on Day7 and Day 14, most samples in this group increased from 180 to 720 folds by day14.

FIG. 24B shows CD8 performed a very similar job as CD3 did in each group: more than half of the samples increased from 100 to 1,270 fold by day 14, the expansion of CD8 in K562hCD3CD28CD137L APCs group are most significant compared to the other group on day14.

FIG. 24C shows CD4 expansion is significant except for the K562hCD3CD28 double APCs group. When compared to K562 control group on day 7 and day 14, triple APCs increased CD4 the most on both day 7 and 14, double beads increased CD4 the second best by day 14. Both double beads and triple APCs groups increased CD4 significantly from day7 to day 14, double beads fold increase from 12 to 780 with an average of 239, triple APCs fold increase from 28-530 at an average of 187.

FIG. 25A shows CD8 TCM increased in each expansion group compared with K562 empty control on day7, triple APCs group is the best and increased from 100-13,000 fold, mean=1684; double beads CD8TCM increased the second best from 25-3,000 fold, mean=811. By the time of day 14, triple beads group still best maintained the level of central memory CD8 T cells.

FIG. 25B shows that every group expanded CD8 TEM compared to K562 empty on day7, triple APCs had most significant expansion; and triple APCs and triple beads continue to increase TEM by day14, most samples from triple APC increased the fold change from 500-15,200.

FIG. 25C shows CD8 TEFF increased in each expansion group on day7, by day 14 all the groups reached at the highest level of CD8 effectors except double APCs did not increase while compared with K562 empty; double beads and triple APCs groups continue increase TEFF from day7 to day14. Triple APCs increased CD8 effectors from 100-1,400 fold and double beads increased from 50-800 on day 14.

FIG. 26A shows T cell fold and CD3 change. Melanoma TILs were cultured with irradiated aAPCs K562hCD3scFvCD28scFvCD137L at 1:1 ratio for 14 days. Cells were counted by automated cell counter at day 0, day 7 and day 14, and divide the day 7 and day 14 live cell numbers by day 0 cell numbers in each expansion as fold increase. Two TIL samples 40040 and 40214 showed significant T cell expansion and CD3 percentage increase by day 14, 100 and 3000 units of hIL2 per ml media did not show a significant difference in CD3 expression.

FIG. 26B shows two identical TIL samples expanded, increased CD8, and showed lower IL2 and higher CD8 in the expansion. The least expanded TIL sample had the most increase in CD4.

FIG. 26C shows the two TIL samples expanded, increased PD1, increased significantly on CD8 and CD4 by day 7, and decreased significantly by day 14 on CD8. The least expanded TIL increased PD1 on both CD4 and CD8 by day 7.

FIG. 27A shows T cell fold change and CD3 expression. The good and the bad TILs both expanded well by day 14, and fewer aAPCs, show better T cell expansion. CD3 has the same tendency; the good TIL CD3 increased the best by day 7, but the bad TIL was catching up by day 14 with the lowest triple APC numbers.

FIG. 27B shows the good TIL had both CD4 and CD8 expanded, lower aAPCs, and better CD8 expanded. The bad TIL had CD8 expanded in terms of numbers of aAPCs.

FIG. 27C shows the two TIL samples increased PD1 the most with the highest numbers of aAPCs by day 7. By the time of expansion, aAPCs numbers did not have an impact on PD1 expression. By day 14, less aAPCs and less PD1 were expressed on both CD8 and CD4.

### DETAILED DESCRIPTION

The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen.

As used herein, the term “antibody or fragments thereof” encompasses chimeric antibodies and hybrid antibodies, with dual or multiple antigen or epitope specificities, and fragments, such as F(ab′)2, Fab′, Fab, scFv, and the like, including hybrid fragments. Thus, fragments of the antibodies that retain the ability to bind their specific antigens are provided. For example, fragments of antibodies which maintain CD3, CD28, CD137, PD1, CTLA4, LAG3, TIM3, BTLA, CD160, 2B4, A2aR, and KIR binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e g immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The disclosed aAPCs can also secrete or express surface bound antibodies or antibody fragments that bind molecules of the T cell inhibitory pathway. In some embodiments, the disclosed aAPCs secrete an antibody or antibody fragment (for example, an scFv) that interferes with suppression of T cells, e.g. by ligation of PD1 with PDL1 such as, for example, use of an anti-PD1 or PDL1 antibody or antibody fragment. This suppression is a normal physiologic immune response meant to prevent over-activation of T cells. However, cancer cells have co-opted this suppression pathway as a means to evade immune recognition and tumor killing. This system is a less costly, more efficient and more rapid alternative to peripheral blood mononuclear cells (PBMCs) and cytokine treatments. The system is less costly because a renewable resource replaces the need for cytokines, antibodies for activation, and PBMC feeders. The faster production time is also clinically meaningful considering that patients have to wait a few months for production of their cells, which can be a difficult task for patients with metastatic cancer. Also, extended culture often produce terminally differentiated T cells that have limited function and persistence when adoptively transferred back into patients. The shorter culture time therefore may allow us to infuse a T cell product that is more physiologic and tumor-reactive. In one aspect, other immune cell inhibitory molecule comprises CTLA4, LAG3, TIM3, BTLA, CD160, 2B4, A2aR, PD-1, ICOS, CD25, TIM3, LAG3, PD1, CD40, CD137, OX40, CD2, LFA-1, CD28, CD154, BTLA, CD160, TIM 1, TIM 4, KIR, any glucocorticoid-induced tumor necrosis factor-related receptor (GITR), and/or any combination thereof. Thus, in one aspect, disclosed herein are methods for expanding an immune cell isolated from a subject for autologous immune therapy, comprising a) providing an artificial antigen presenting cell (aAPC) comprising a cell having a membrane, wherein the cell secretes one or more single chain variable fragment (scFv) antibodies that bind a T cell inhibitory molecule, or a combination thereof, wherein the cell contains on its membrane: one or more scFv that selectively bind an immune cell selective receptor (such as, for example CD3) and one or more scFv or ligands that bind a co-stimulatory molecule on T-cells (such as, for example, CD28 and/or 4-1BB); and b) contacting the isolated immune cell with an effective amount of the aAPC to expand the immune cell in an amount effective for immunotherapy. For example, in one aspect disclosed herein are methods for expanding an immune cell isolated from a subject for autologous immune therapy, comprising a) providing an artificial antigen presenting cell (aAPC) comprising a cell having a membrane, wherein the cell secretes one or more single chain variable fragment (scFv) antibodies that bind a T cell inhibitory molecule, or a combination thereof, wherein the cell contains on its membrane: one or more scFv that selectively bind CD3 and one or more scFv or ligands that bind CD28 and/or 4-1BB (such as, for example an anti-CD38 scFv and/or 4-BBL); and b) contacting the isolated immune cell with an effective amount of the aAPC to expand the immune cell in an amount effective for immunotherapy.

In one aspect, the methods of expanding immune cells can be used for expanding TILs or MILs for use in immunotherapy. It is understood and herein contemplated that the use of said cells can comprise expanding Tils or Mils form a tissue from a subject. In one aspect, the TILs or MILs may be obtained from any tissue (such as, for example, biopsy, blood, urine, sputum, saliva, tissue lavage) in a subject by any means known in the art (tissue resection, biopsy phlebotomy, core biopsy). Because the tissue sample can be used, it can be advantageous to screen expanded TILs or Mils for desired activity (such as, for example, tumoricidal activity via expression of CD107). Thus, in one aspect, disclosed herein are methods for expanding tumor infiltrating lymphocytes for use in immunotherapy, comprising a) providing an artificial antigen presenting cell (aAPC) comprising a cell having a membrane, wherein the cell secretes one or more single chain variable fragments (scFv) that bind a T cell inhibitory molecule, or a combination thereof, wherein the cell contains on its membrane one or more scFv that selectively bind CD3 and one or more scFv or ligand that binds a co-stimulatory molecule on T-cells; b) expanding tumor infiltrating lymphocytes (TILs) from a biopsy of a tumor from a subject; c) screening the TILs for tumoricidal activity using flow cytometry to detect CD107 expression; and d) contacting the tumoricidal TILs with an effective amount of the aAPC to expand the tumoricidal TILs. In one aspect, the disclosed methods can further comprise infusing the expanded tumoricidal TILs into the subject in an effective amount to treat the tumor.

It is understood and herein contemplated that the expansion of immune cells (such as, for example T cells, NK cells, or B cells) including TILs and MILs can occur ex vivo, in vitro, or in situ with the expansion occurring outside the subject and administration occurring after expansion. However, it is understood and herein contemplated that the expansion of immune cells including TILs and MILs can also occur in vivo by directly administering aAPC comprising an scFc that binds to a T cell inhibitory molecule, and an scFv recognizing an immune cell receptor (such as, for example) CD3 and scFv or ligands binding to co-stimulatory molecules (such as, CD28 and 4-1BB) directly to the subject in need of treatment.

In one aspect, the aAPC can further comprise on its membrane surface expression of a scFv or ligand that specifically binds a cytokine such as, IL2R, IL7R, IL12R, IL15R, IL18R, IL10R, or any combination thereof.

The aAPC can be derived from any antigen presenting cell including a cell line such as, for example K562, NIH/3T3, Chinese hamster ovary (CHO), or Human Embryonic Kidney (HEK) cell line.

It is understood and herein contemplated that the disclosed methods can result in an expanded immune cell. Accordingly, in one aspect disclosed herein are immune cells produced by any method for expanding immune cells disclosed herein.

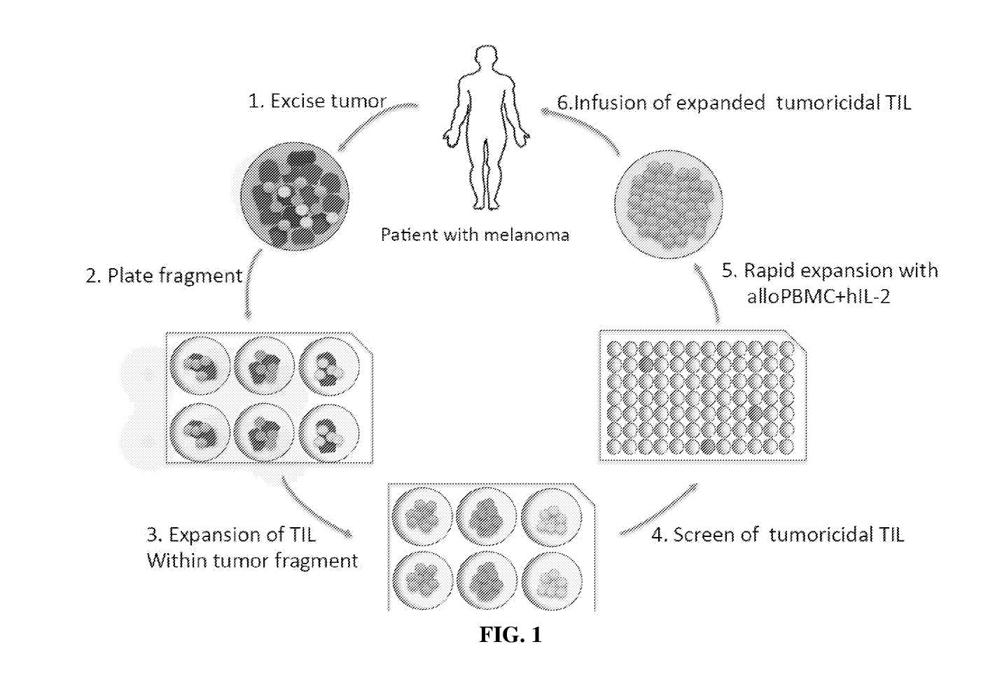

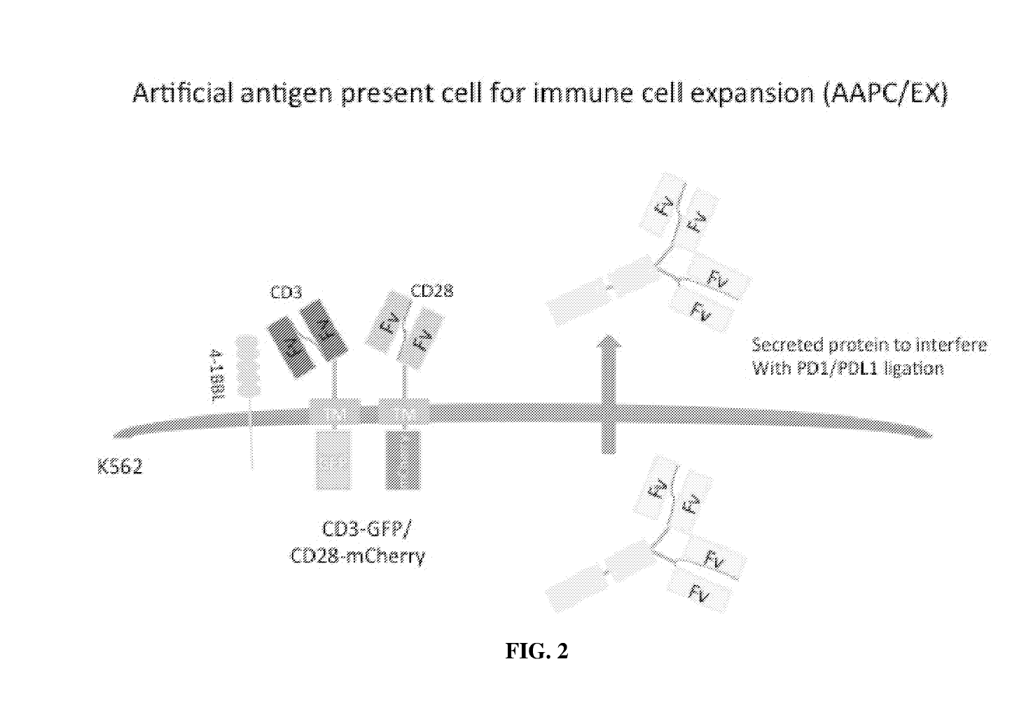

Turning to FIG. 1, a prior art method for expanding tumor infiltrating lymphocytes (TILs) involves obtaining tumor fragments from the patient by surgery, and directly incubating tumor fragments in culture plates in a complete media (e.g. RPMI 1640, 100 U/mL penicillin, 100 μg/mL streptomycin, 2 mmol/Lglutamine, supplemented with 10% human serum). A dense carpet of lymphocytes appears around the tumor fragment after 1-2 weeks of incubation. Alternatively, the tumor fragments are digested enzymatically by collagenase, hyaluronidase, and DNAse, followed by purification on a single step ficoll gradient. The TILs fraction is separated by flow cytometery based on CD107 expression and incubated in the complete medium. These lymphocytes (young TILs) are expanded to a confluent growth. While the young TILs expand, they eliminate tumor cells by direct contact or by the secretion of cytokines. Each TILs culture from initial tumor fragment gives around 5×10⁷ cells after 21-36 days of culture. TILs are then rapidly expanded in presence of anti-CD3 antibody and IL-2 in culture flask/bag. This generally involves TIL culture in which irradiated peripheral blood mononuclear cells (PBMCs) serve as feeder cells along with anti-CD3 antibody. The expansion step increases the cell count by 3000 folds in about 14 days. This process requires a large quantity of cytokines and growth factors.

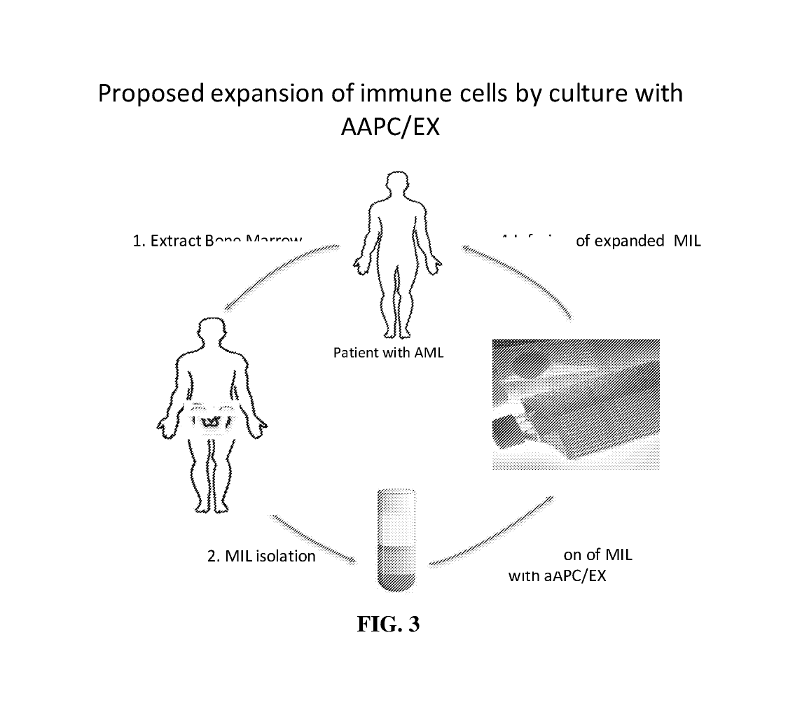

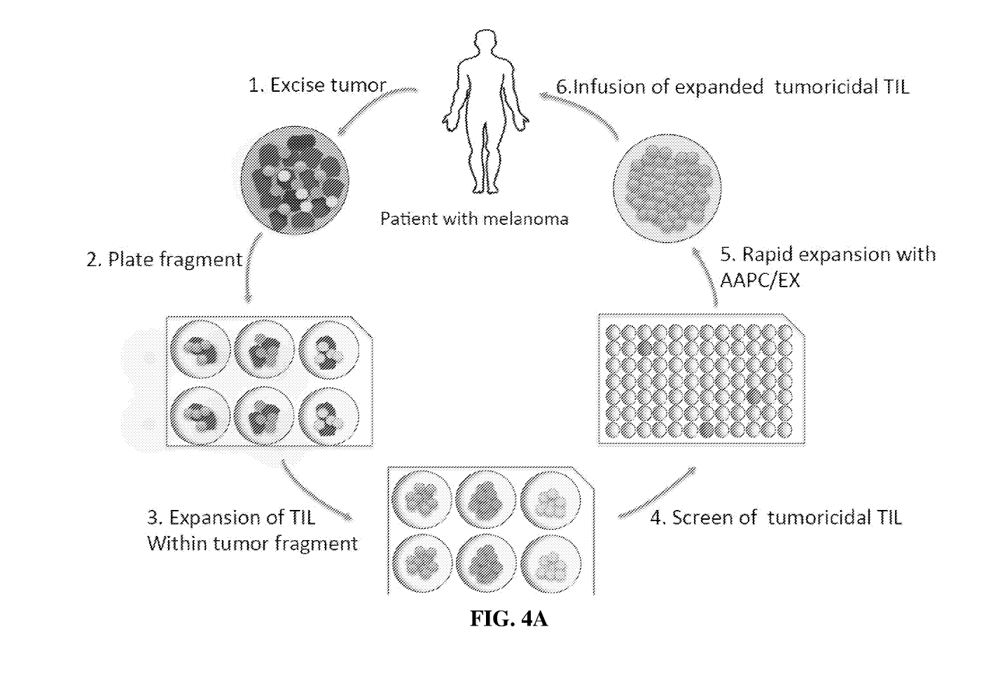

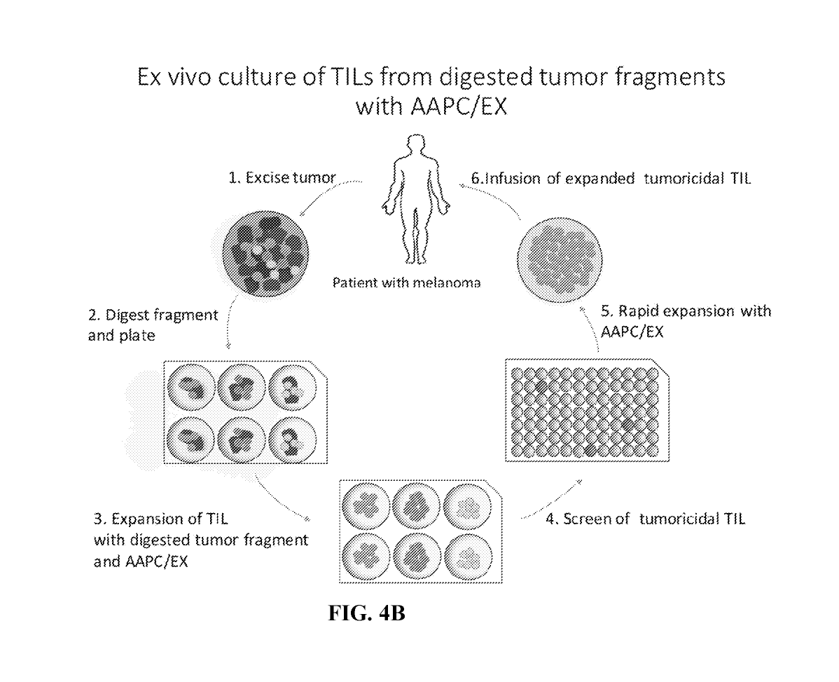

Turning to FIG. 4, PBMCs can be replaced with the disclosed artificial antigen presenting cells (aAPCs) to rapidly expand the TILs from melanoma fragments (FIG. 4B) or as replacement for allogeneic PBMC feeders (FIG. 4A). Likewise, as shown in FIG. 3, aAPCs can be used to expand marrow-infiltrating lymphocytes (MILs) and other immune cells for immunotherapy.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Expansion of Immune Cells with aAPC/EX

Protocol

1. Isolate T Cells from Bone Marrow and Whole Blood from Patients (Day0)

a. Dilute BM and PBMCs with 1× DPBS at 1:1 ratio, add 35 ml of diluents onto top of Ficoll (13-15 ml) slowly in 50-ml conical tubes, then centrifuge at 1500 RPM with break off for 20 minutes at 18-200 C.

b. Carefully collect the lymphocytes monocytes layer and wash the cells with 1× PBS twice.

c. Taking the cells above for isolating T cells by using Human T Cell Enrichment Kit (catalog #19051, STEMCELL Technologies Inc) and re-suspend T cells in 10% FBS of AIM-V medium and cells are ready for use.

2. Preparation of aAPC/EX Cells (Day0)

a. K562 cells were transduced with retrovirus, which express hCD3scFv, hCD28scFv and h4-1BBL, or express hCD3scFv and hCD28scFv.

b. Gene transduced K562 cells were cultured in 10% FBS of RMPI media, spin down the cells at 1500 rpm for 5 minutes and count the cells, irradiate the cells at a dosage of 100 Gy.

### 3. T Cell Phenotype Analysis and T Cell Expansion (Day0-Day14)

a. Day0, using aliquots of the T cells from Bone Marrow and PBMCs for 15 color panel of flow staining, which looks into the expression of PD1, TIM3, LAG3, CD25, CD69, 4-1BB and CD69 on CD4 and CD8, or central memory and effector memory CD4 and CD8 population initially and also continues investigating the dynamic change of activation and exhaustion markers during T cell expansion.

b. Day0, seed the human T cells and the irradiated gene transduced K562 cells or K562 empty cells as a negative control into 96-well, or 24-well plates for T cell stimulation and expansion. The ratio of T cells versus K562 cells is 2:1 or 3:1. The cell culture media is 10% FBS and 30 IU human IL-2/ml.

c. For a comparison between traditional T cell expansion and K562 CAR aAPCs, Dynabeads Human T-Activator CD3/CD28 were incubated with T cells as the same procedure as aAPCs on day0 (Dynabeads catalogue #1131D, gibco by Life technologies).

d. Incubate the cells in a 37° C. and 5% CO2 tissue culture incubator.

e. On day4, day7 and day11, discard half of old media, add irradiated aAPCs and irradiated K562 empty cells into the co-culture with 10% FBS and 30 IU human IL-2/ml AIM-V medium; Dynabeads only add on day7 with the same medium.

f. Day7, count and collect portion of expanded T cells for Flow staining, add aAPCs and beads as described above. On day14, the same cell counting and Flow staining were performed as well.

Results

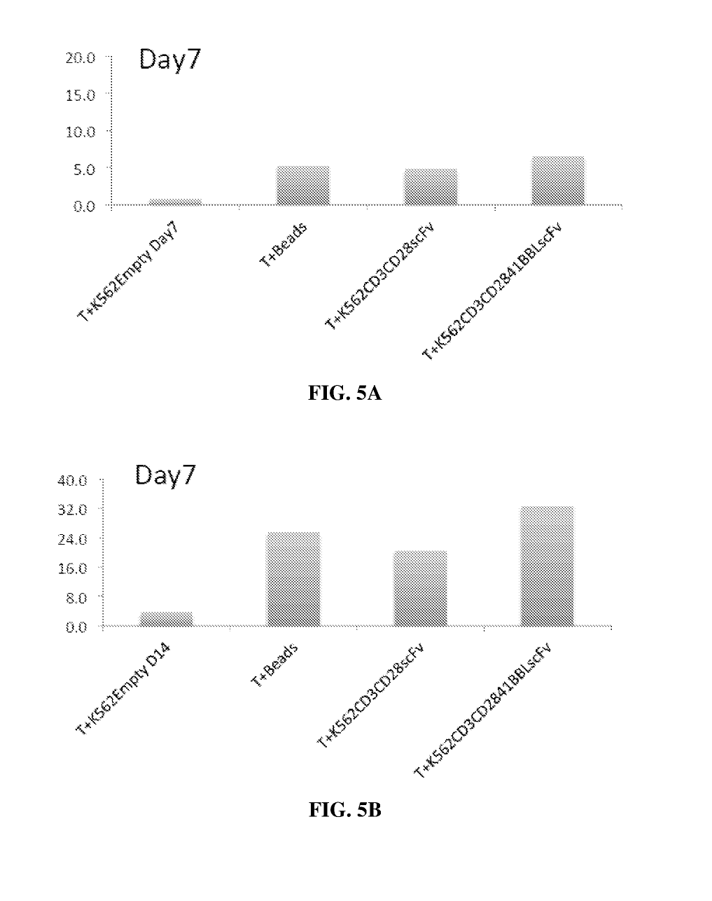

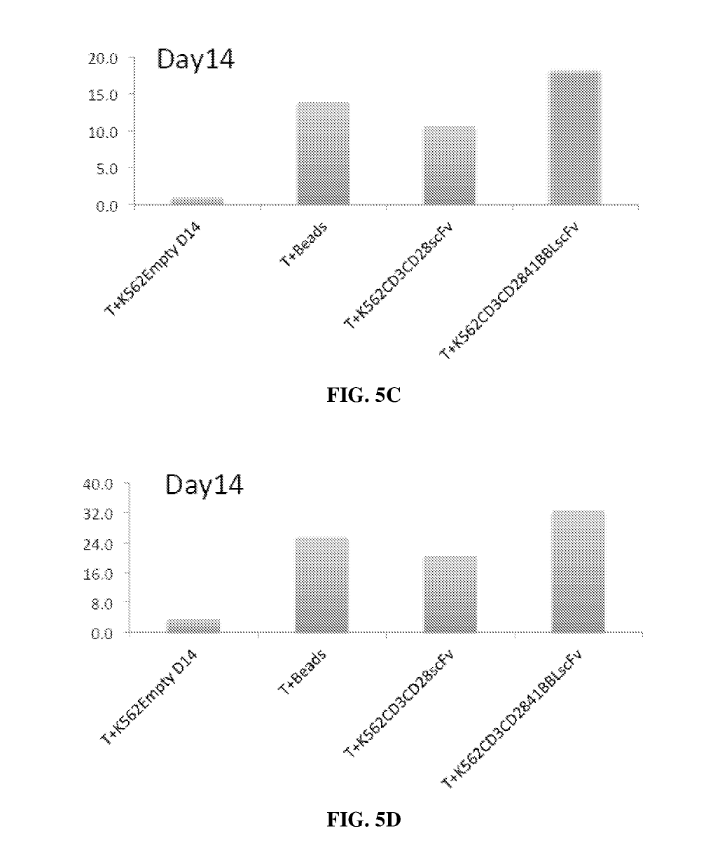

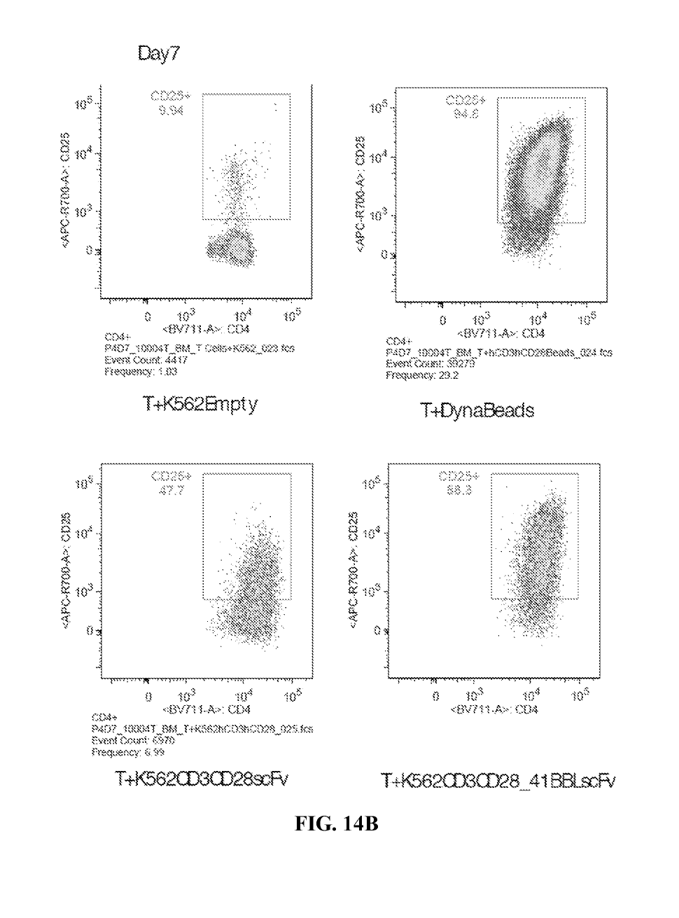

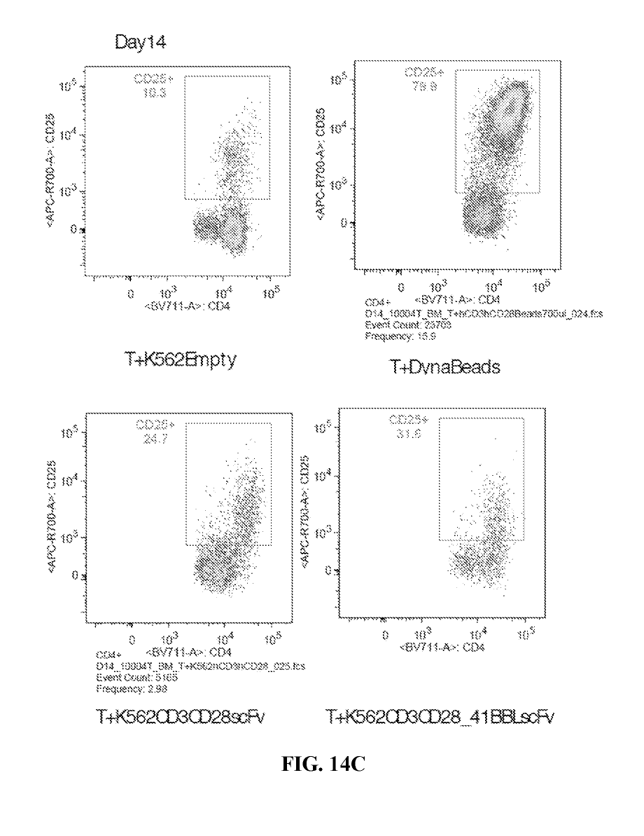

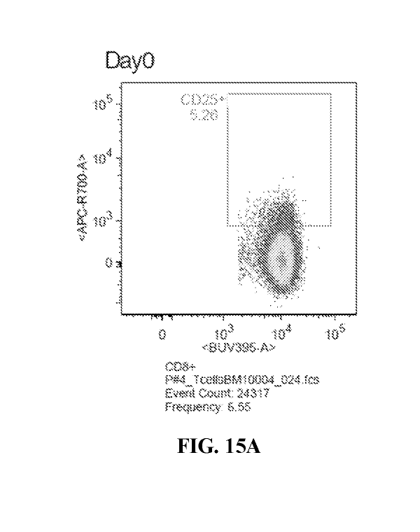

FIGS. 5A to 5D show fold change of live (FIGS. 5A and 5C) and total (FIGS. 5B and 5D) bone marrow T (BM-T) cells after stimulation with Dynabeads® or aAPCs for 7 days (FIGS. 5A and 5B) and 14 days (FIGS. 5C and 5D).

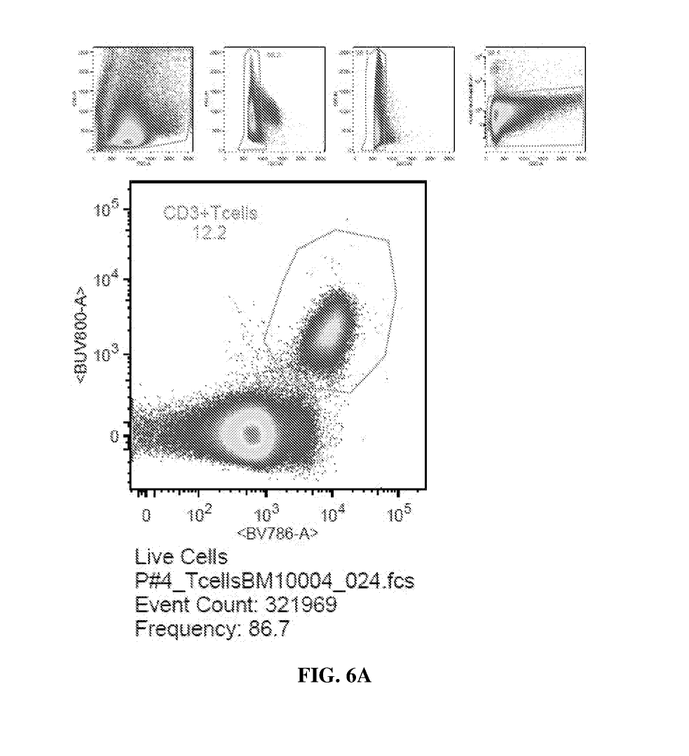

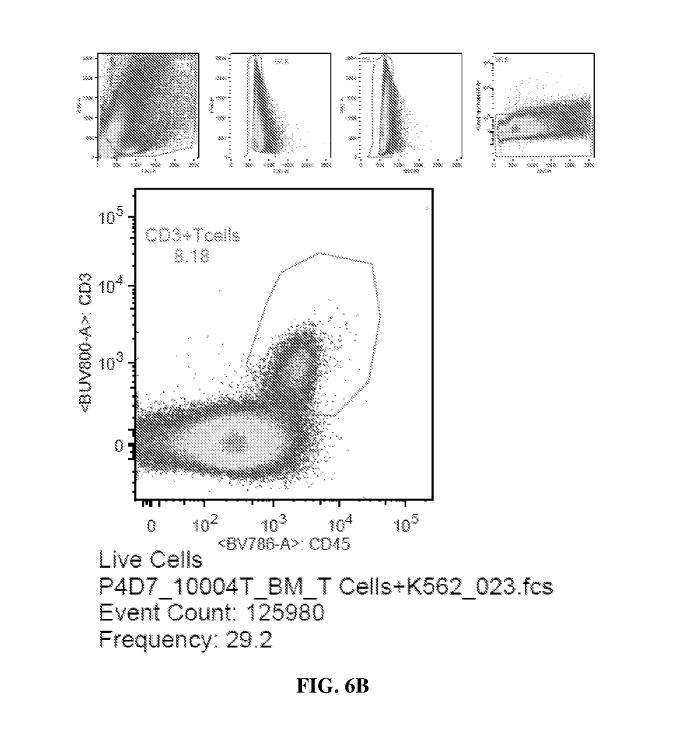

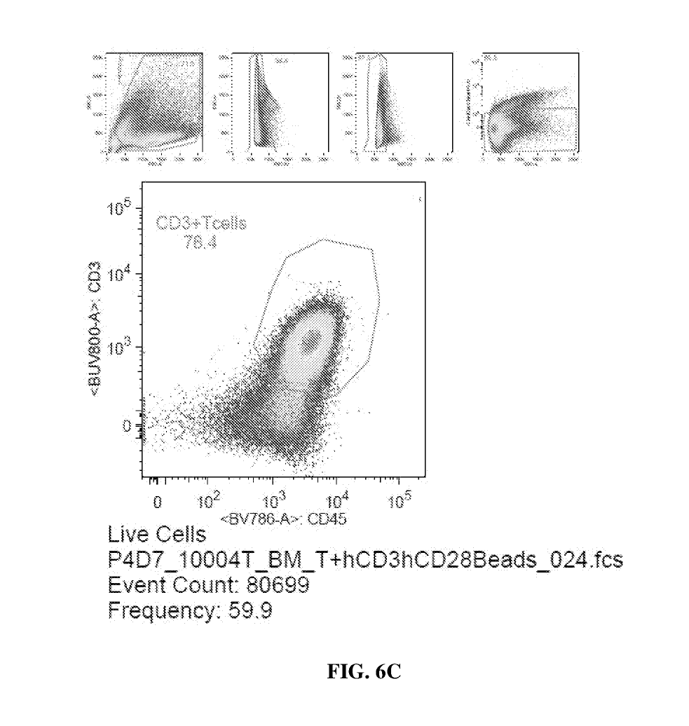

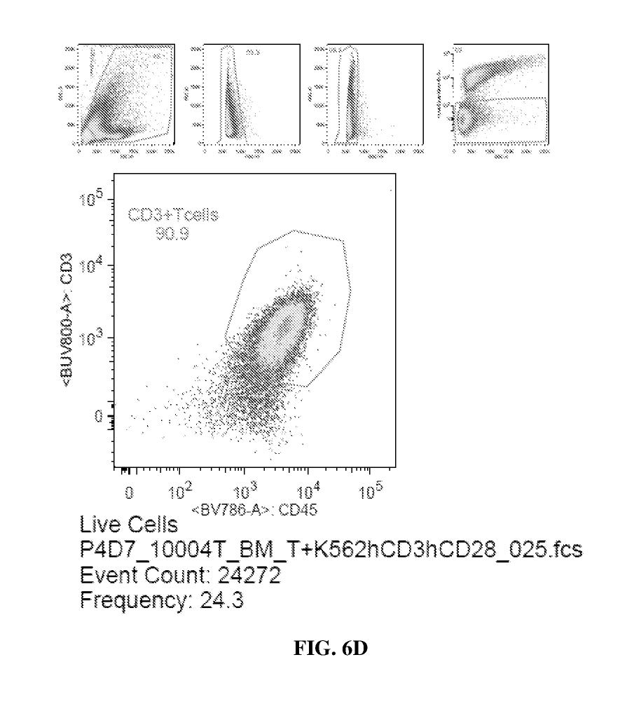

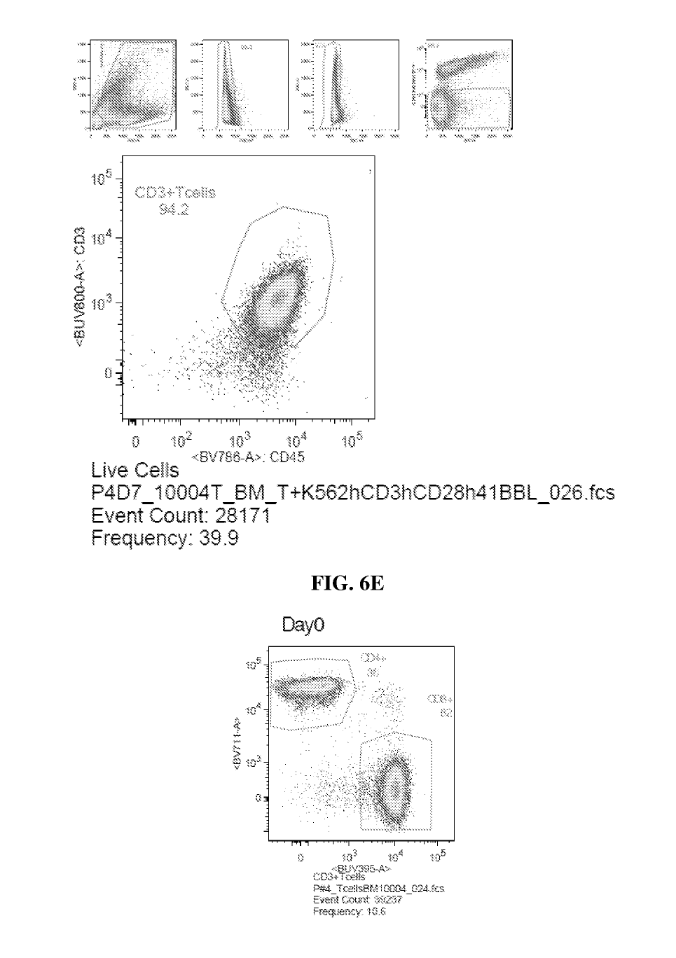

FIG. 6A to 6E show gating strategy for counting live CD3+ T cells stimulation with Dynabeads® or aAPCs.

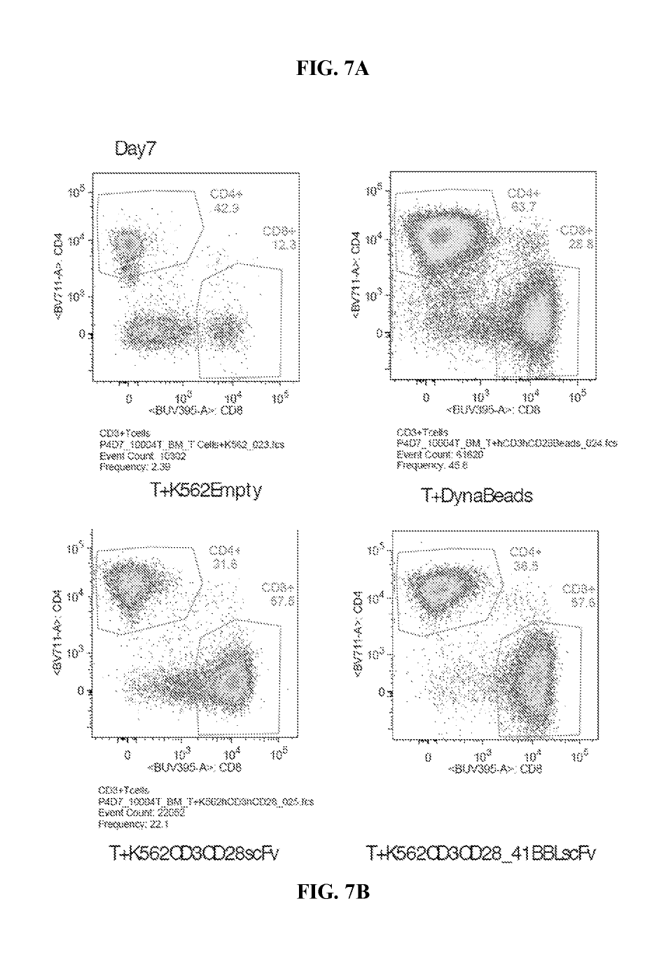

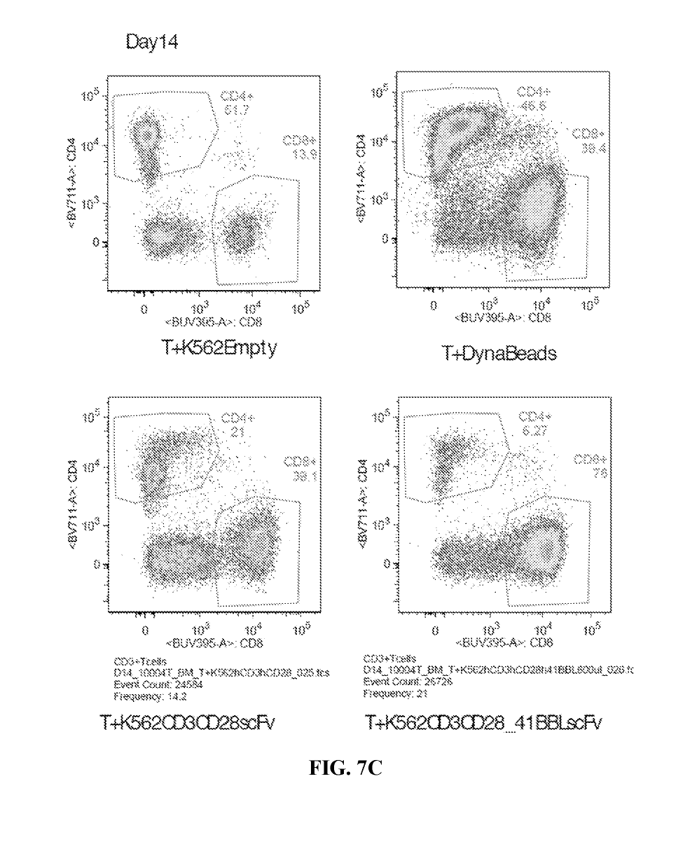

FIGS. 7A to 7C show gating strategy for counting CD3+/CD4+ and CD3+/CD8+ cells after stimulation with Dynabeads® or aAPCs. APCs containing 4-1BBL continued to increase CD8 and decrease CD4 populations until Day14.

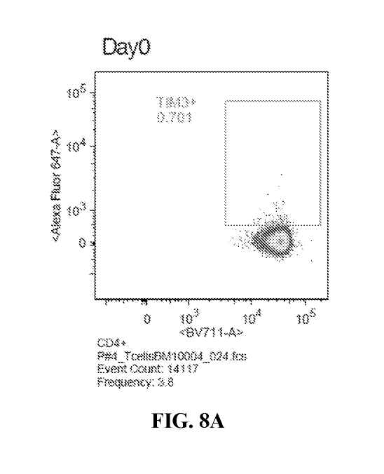

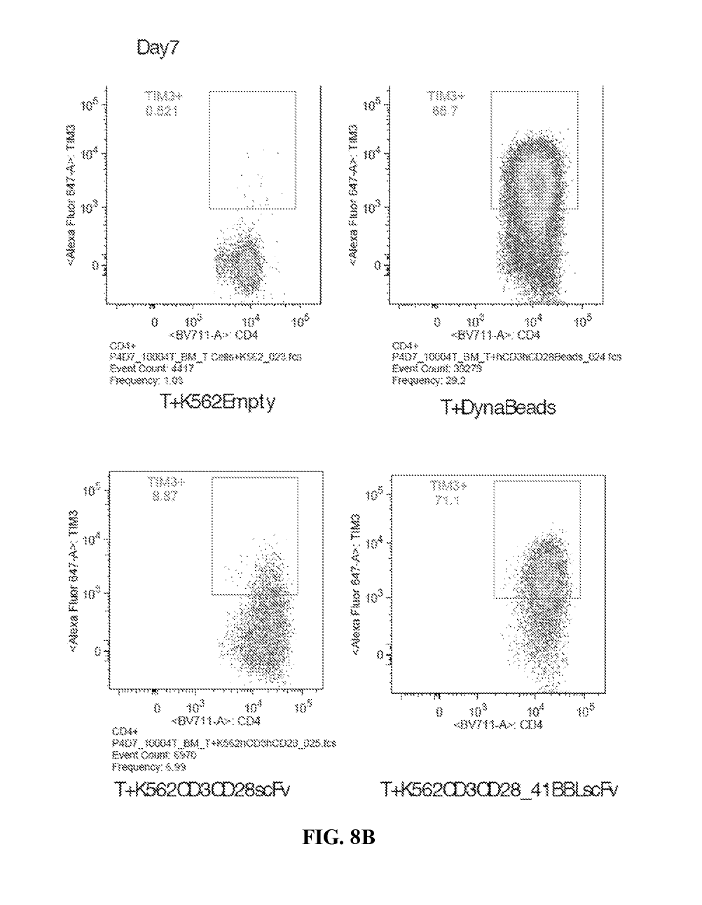

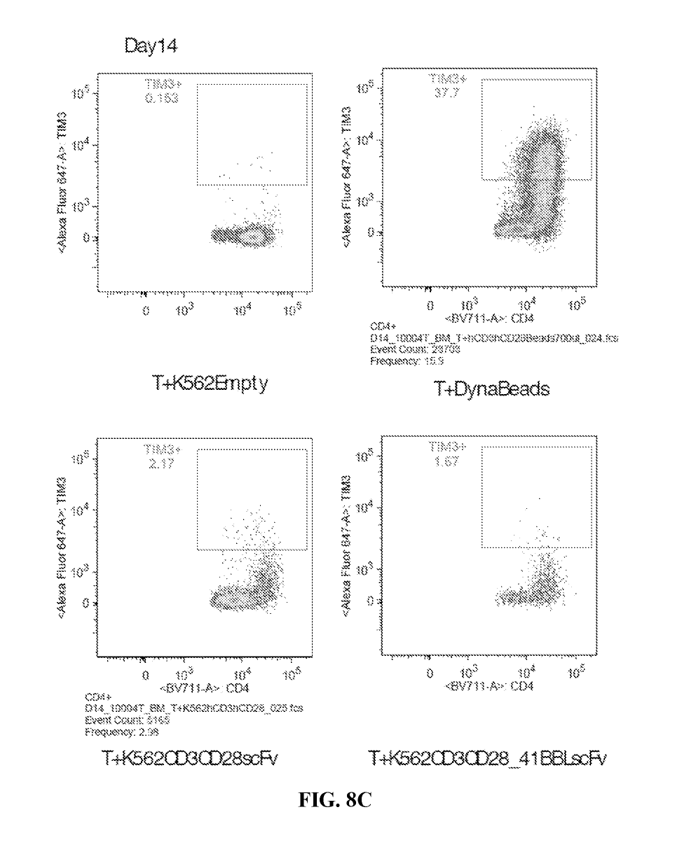

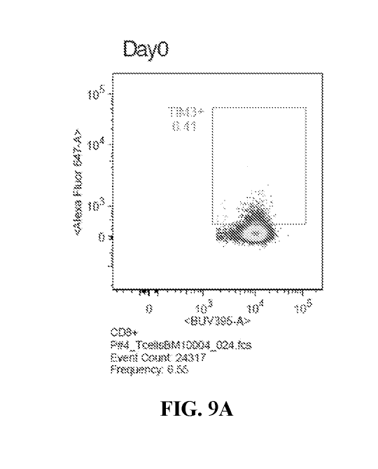

FIGS. 8A to 8C show gating strategy for counting CD4+/TIM3+ cells after stimulation with Dynabeads® or aAPCs.

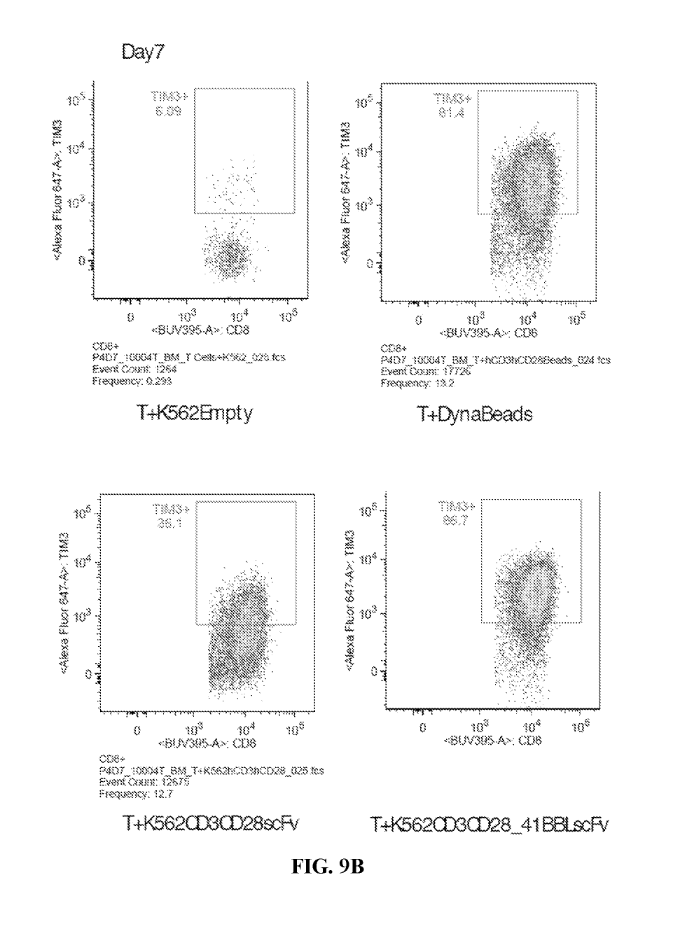

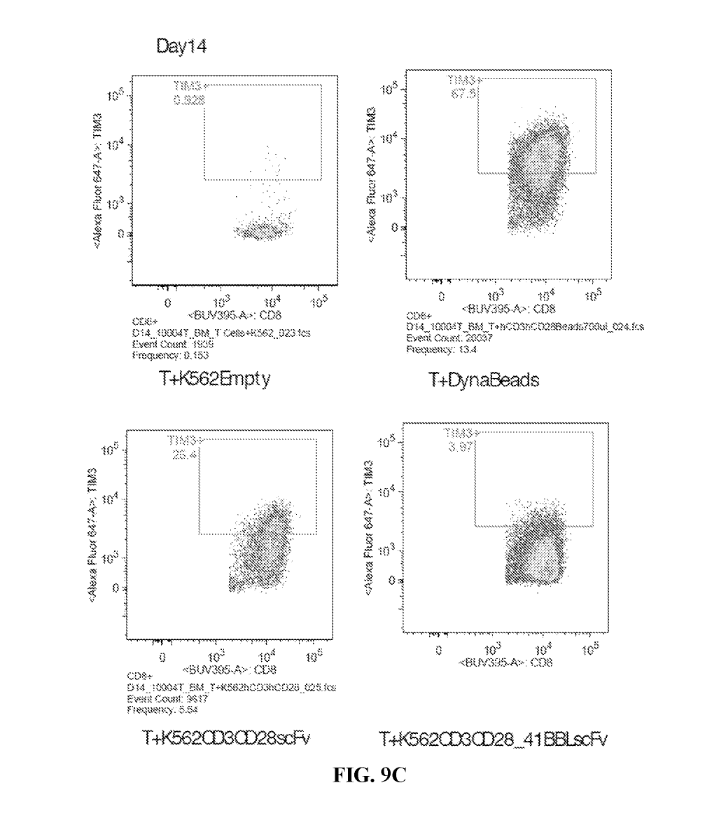

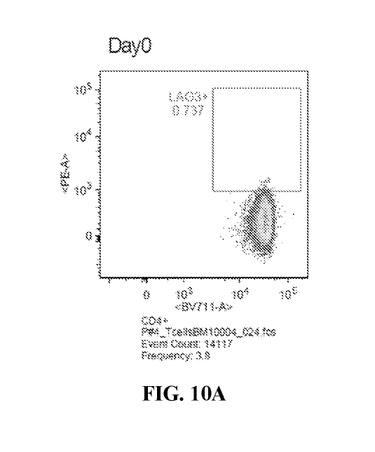

FIGS. 9A to 9C show gating strategy for counting CD8+/TIM3+ cells after stimulation with Dynabeads® or aAPCs. APCs containing 4-1BBL dramatically dropped TIM3 on both CD4 and CD8 cells by day 14.

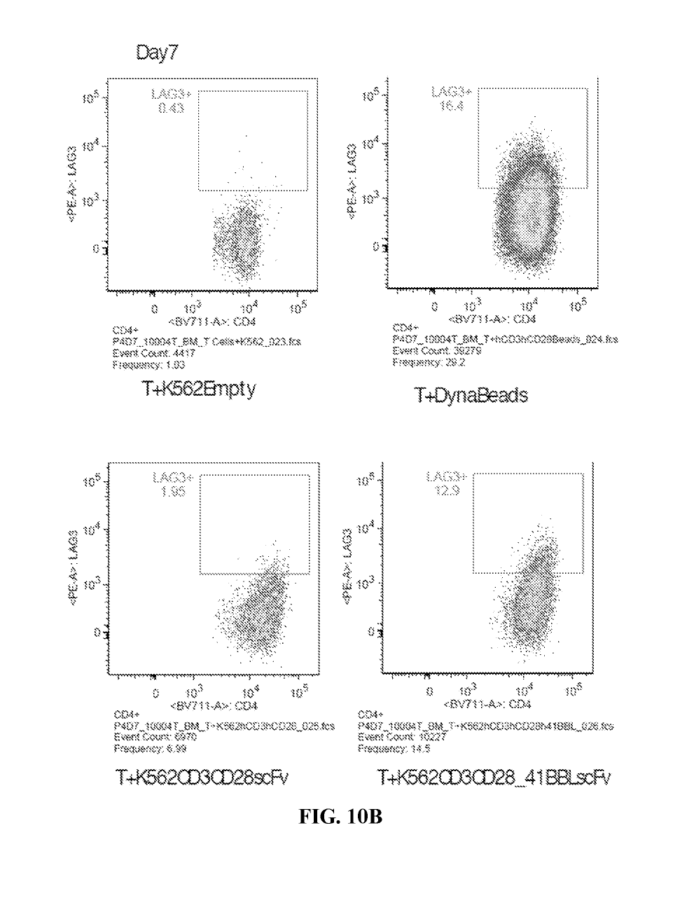

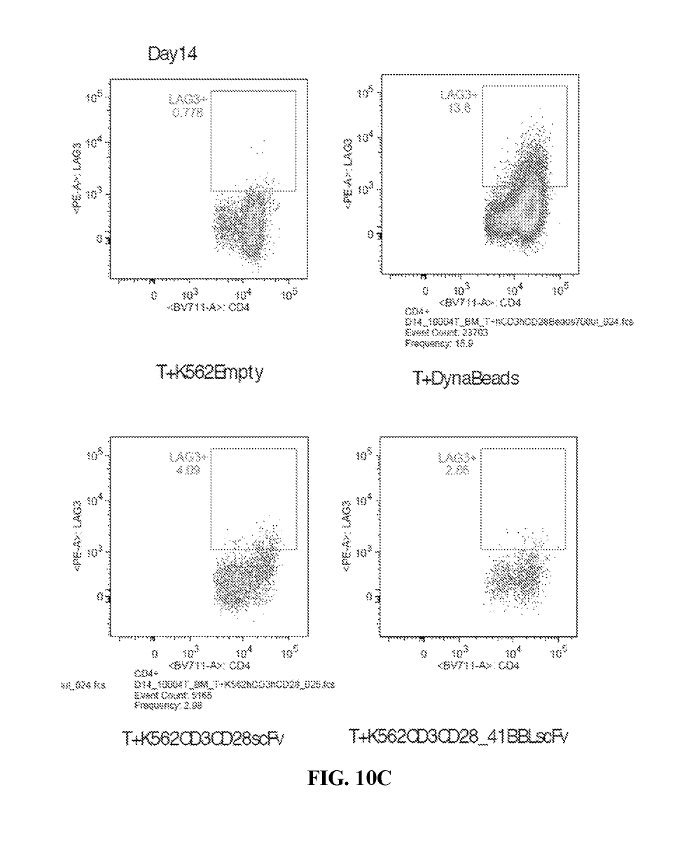

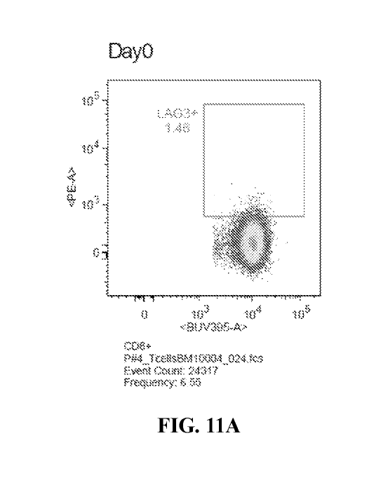

FIGS. 10A to 10C show gating strategy for counting CD4+/LAG3+ cells after stimulation with Dynabeads® or aAPCs.

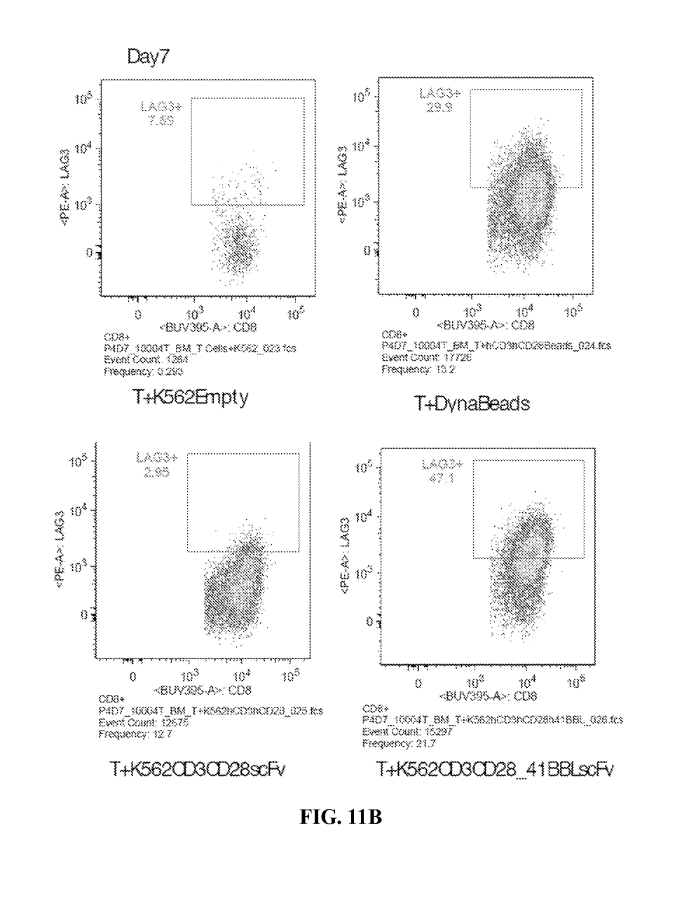

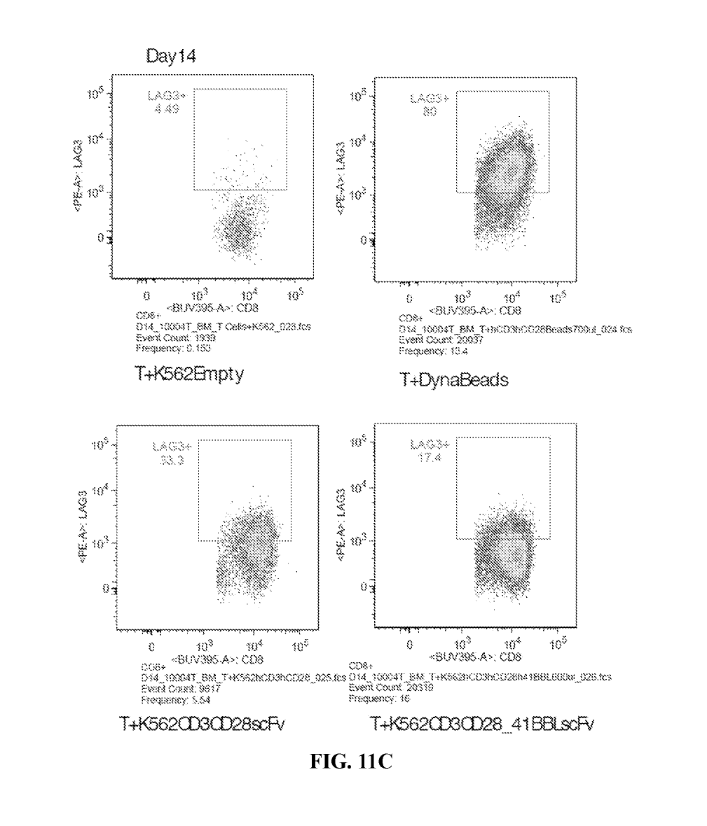

FIGS. 11A to 11C show gating strategy for counting CD8+/LAG3+ cells after stimulation with Dynabeads® or aAPCs. APCs containing 4-1BBL dramatically dropped LAG3 but increased in beads and CD3CD28scFv APC groups on CD8 by day 14.

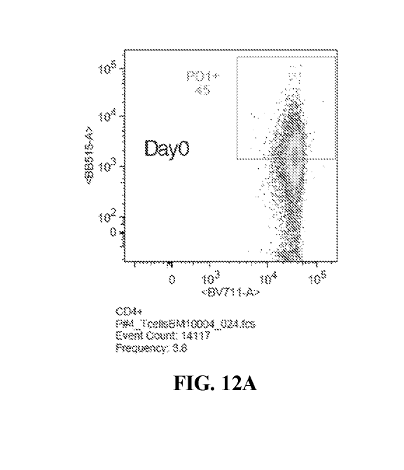

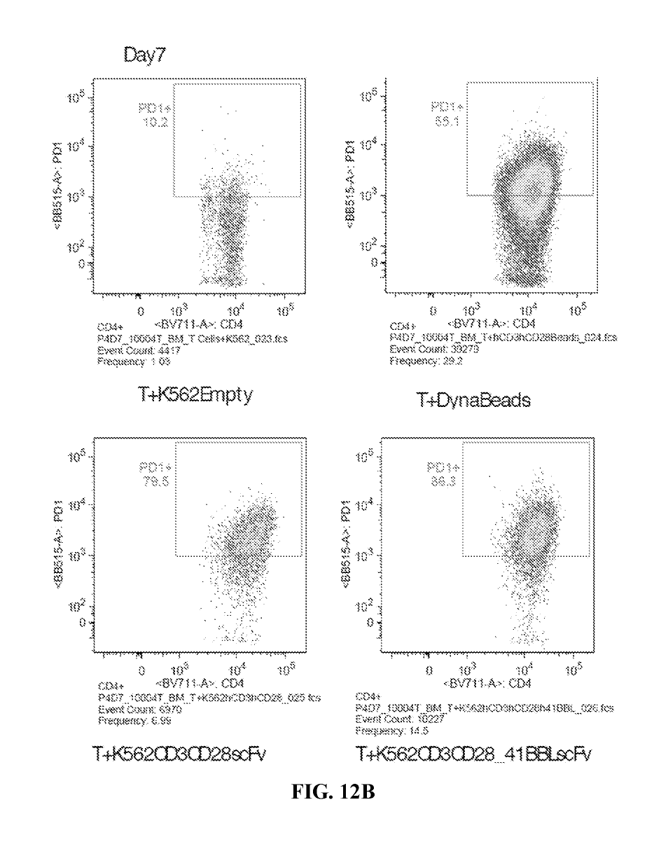

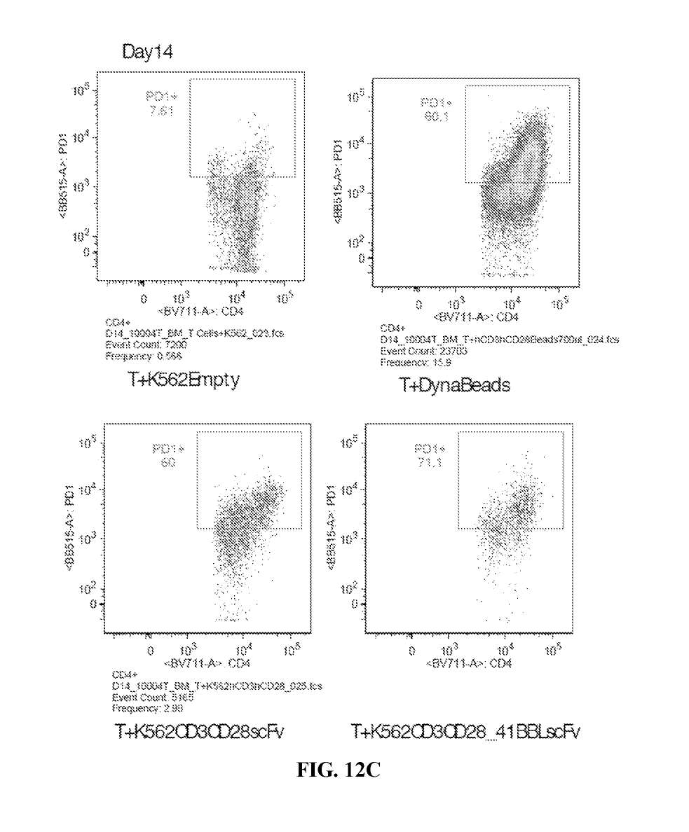

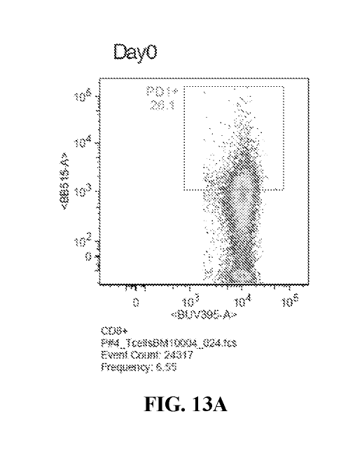

FIGS. 12A to 12C show gating strategy for counting CD4+/PD1+ cells after stimulation with Dynabeads® or aAPCs.

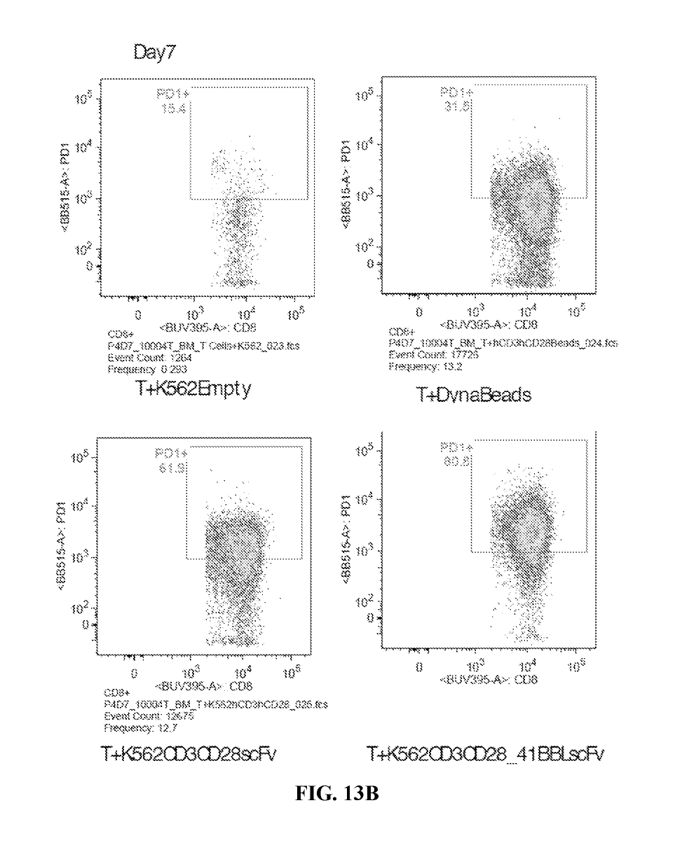

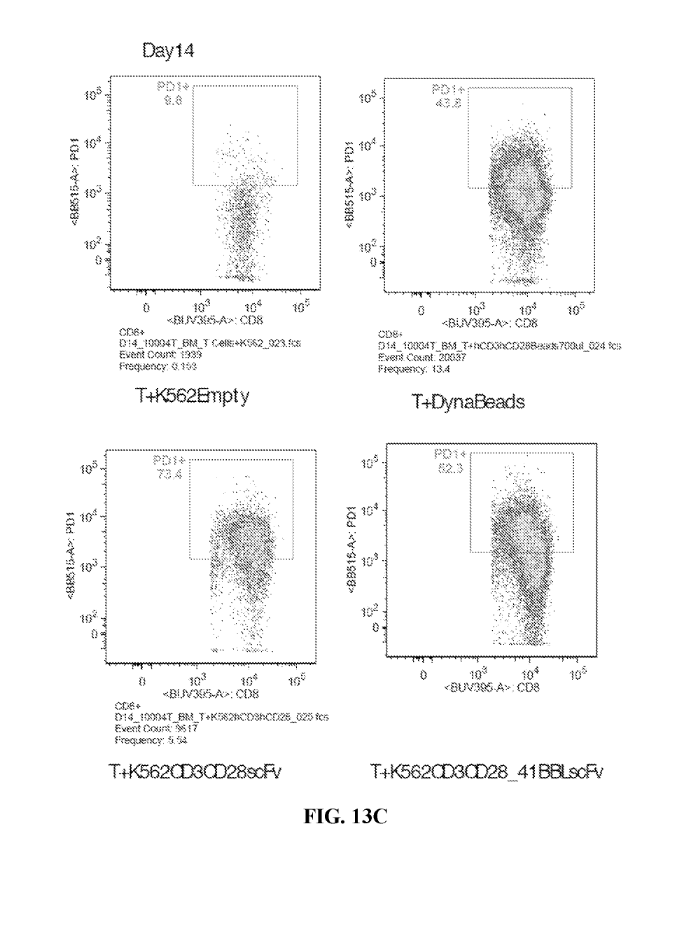

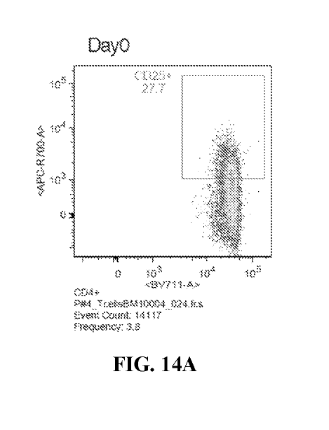

FIGS. 13A to 13C show gating strategy for counting CD8+/PD1+ cells after stimulation with Dynabeads® or aAPCs. APCs containing 4-1BBL showed the same phenomenon as LAG3.

FIGS. 14A to 14C show gating strategy for counting CD4+/CD25+ cells after stimulation with Dynabeads® or aAPCs.

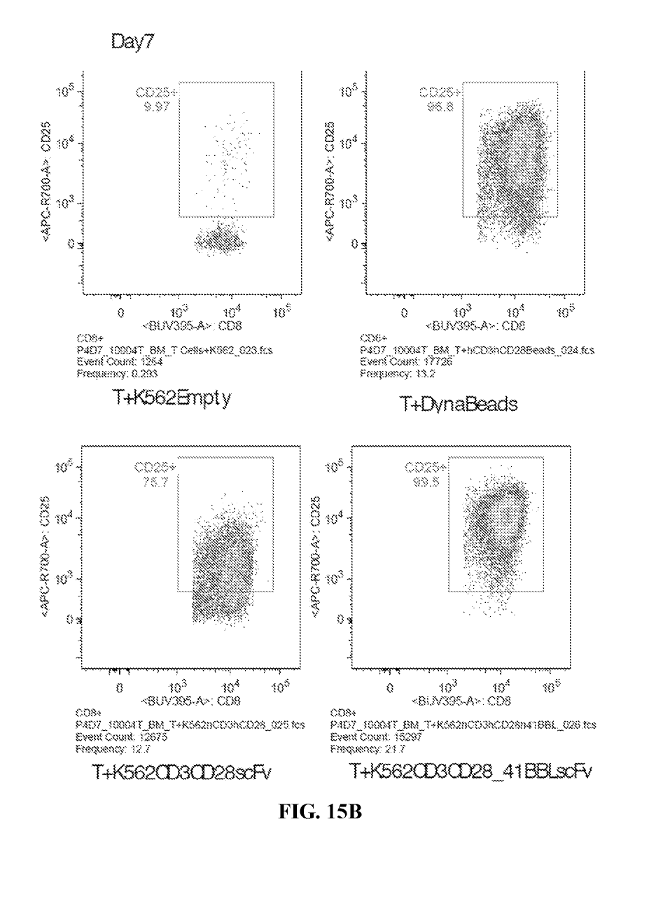

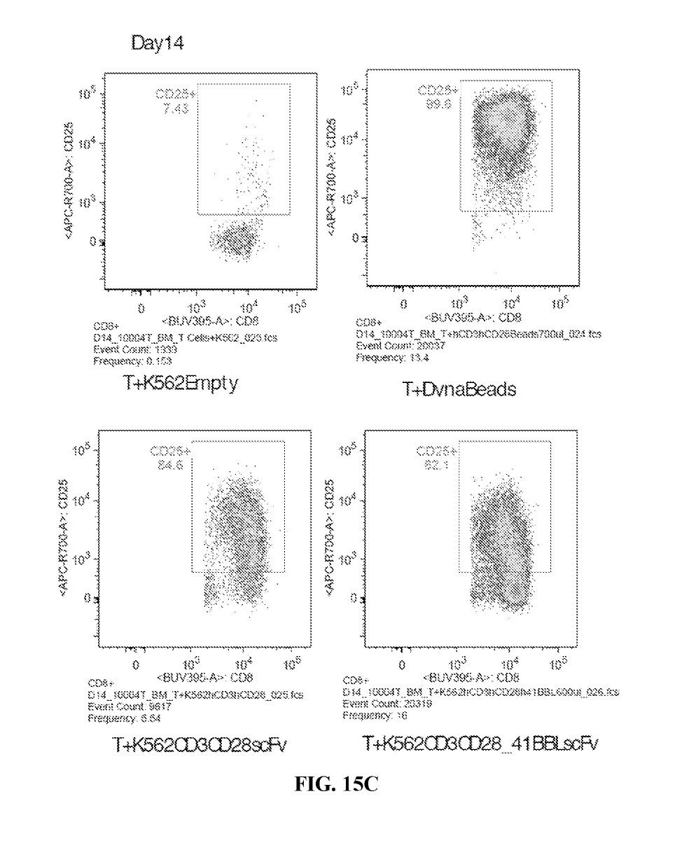

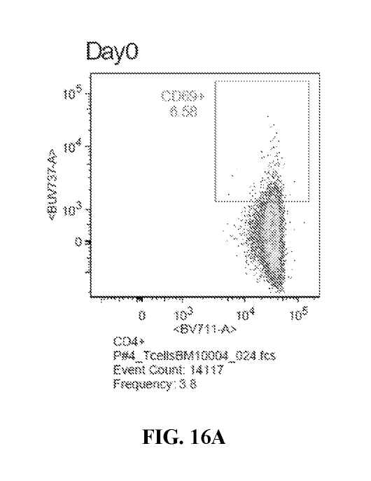

FIGS. 15A to 15C show gating strategy for counting CD8+/CD25+ cells after stimulation with Dynabeads® or aAPCs. All groups decreased CD25 on CD4 cells, but APCs containing 4-1BBL decreased the most. Only APCs containing 4-1BBL decreased CD25 on CD8 by day 14.

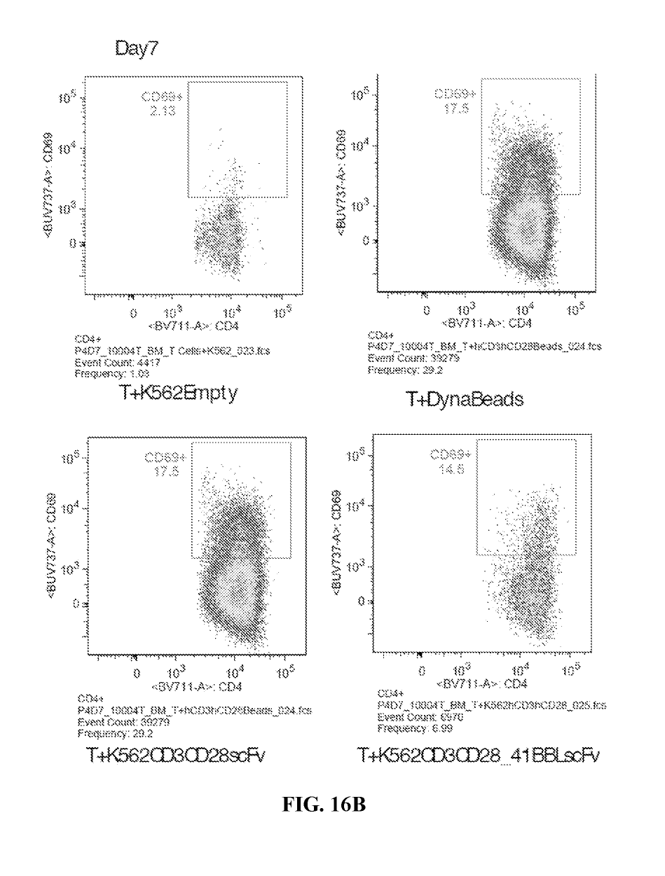

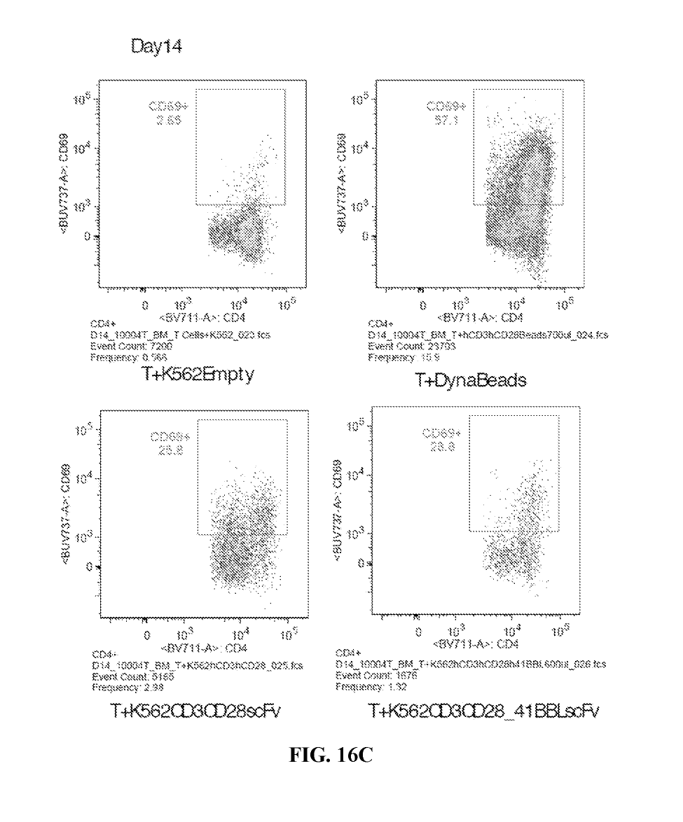

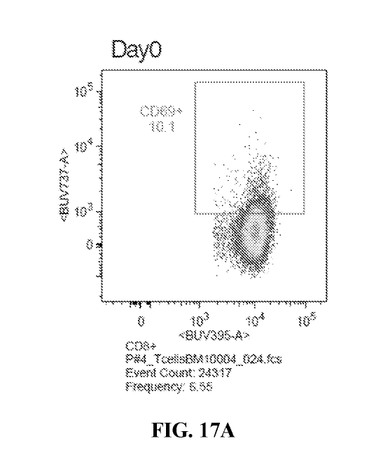

FIGS. 16A to 16C show gating strategy for counting CD4+/CD69+ cells after stimulation with Dynabeads® or aAPCs.

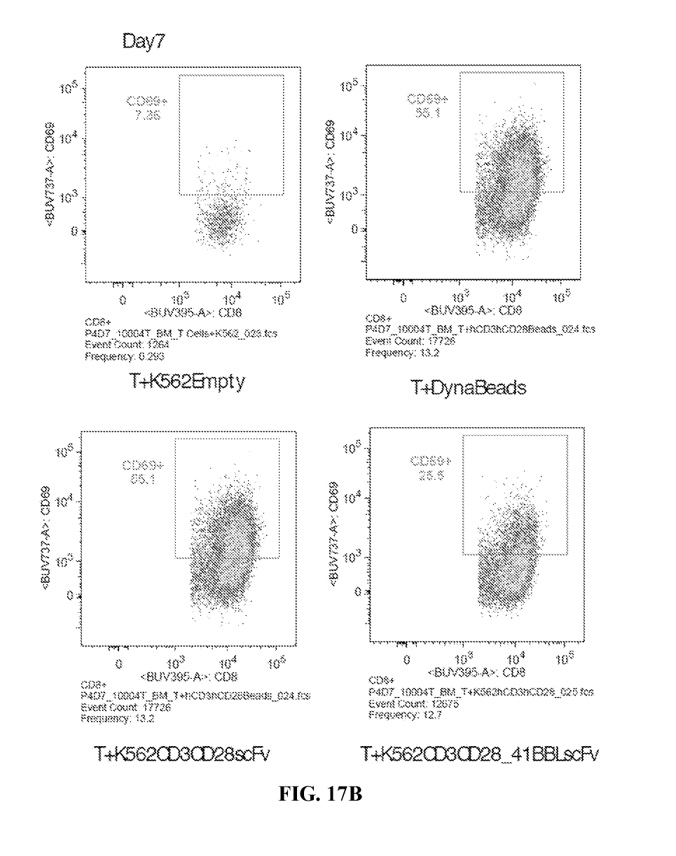

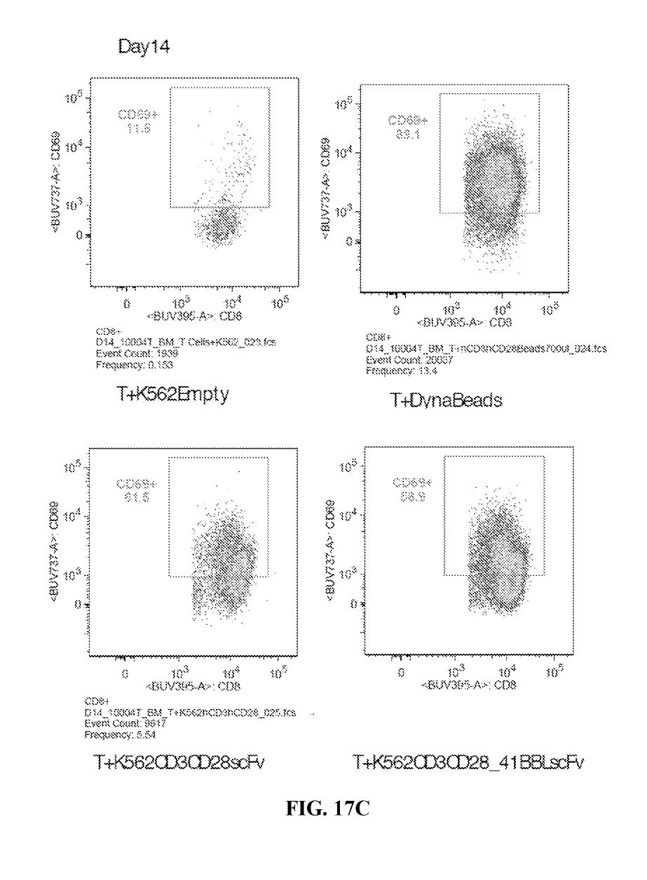

FIGS. 17A to 17C show gating strategy for counting CD8+/CD69+ cells after stimulation with Dynabeads® or aAPCs. All groups increased CD69, especially on CD8 cells.

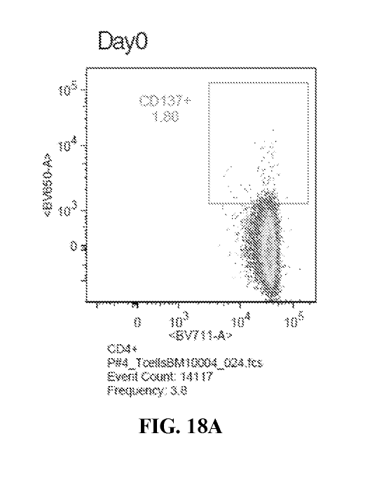

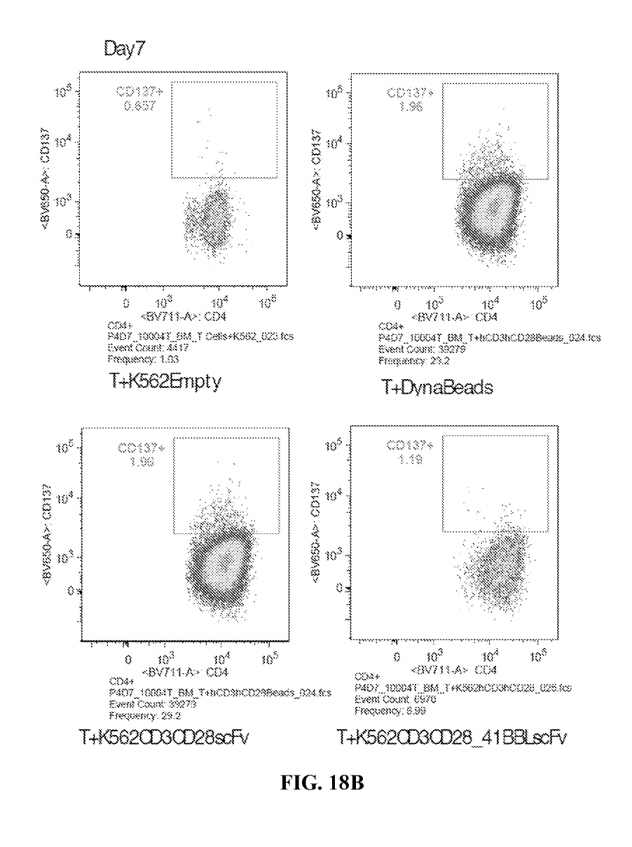

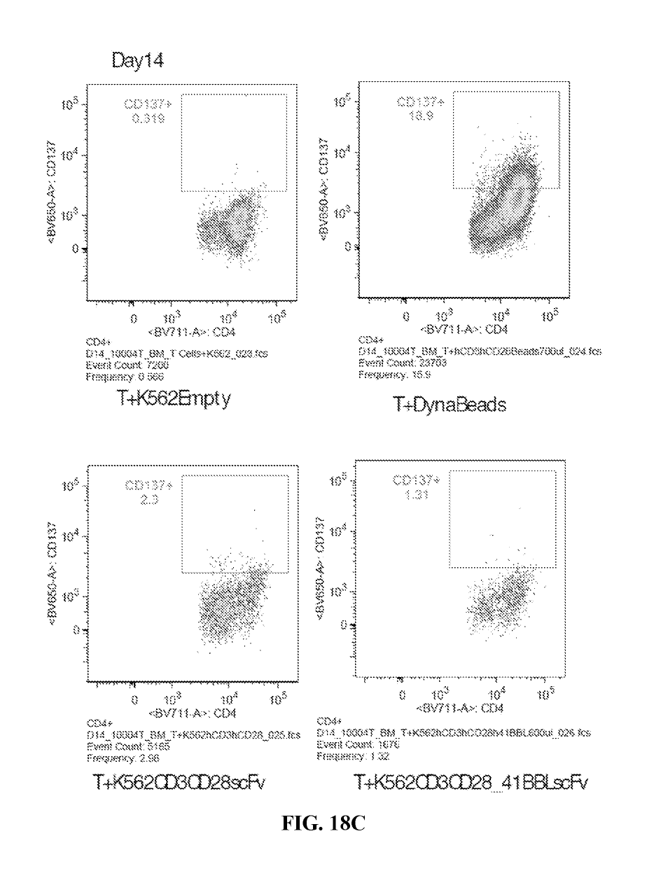

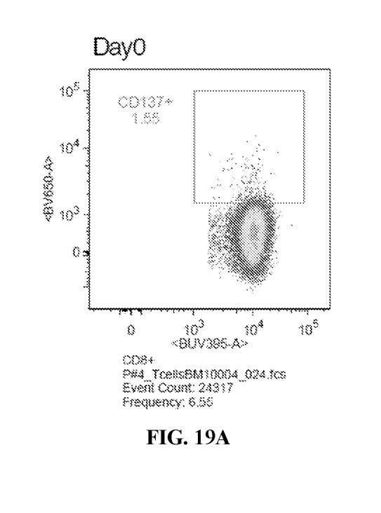

FIGS. 18A to 18C show gating strategy for counting CD4+/CD137+ cells after stimulation with Dynabeads® or aAPCs.

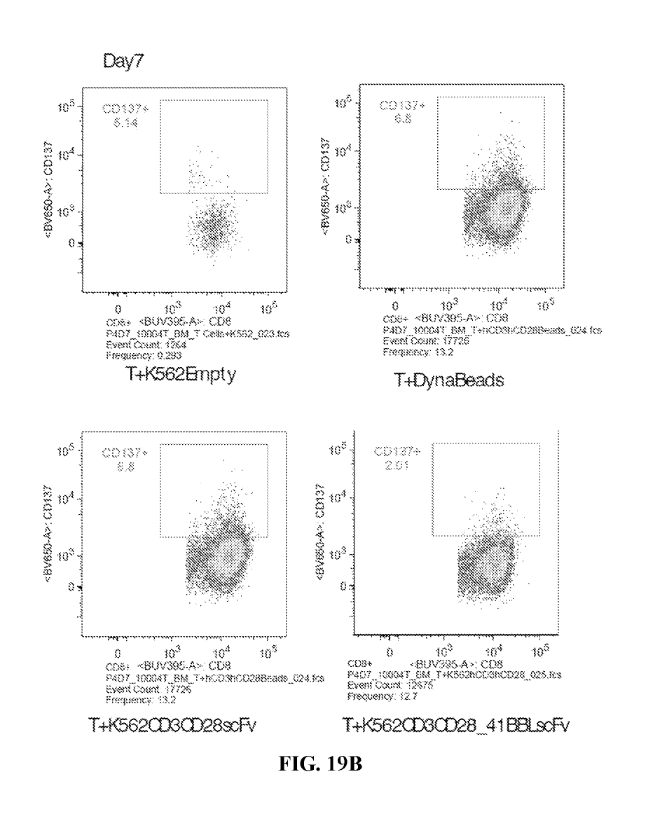

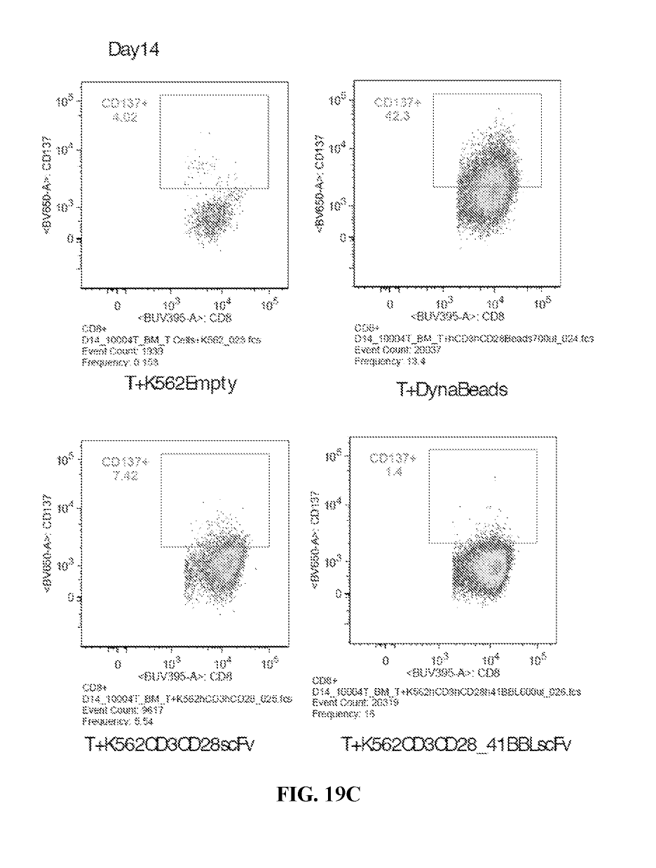

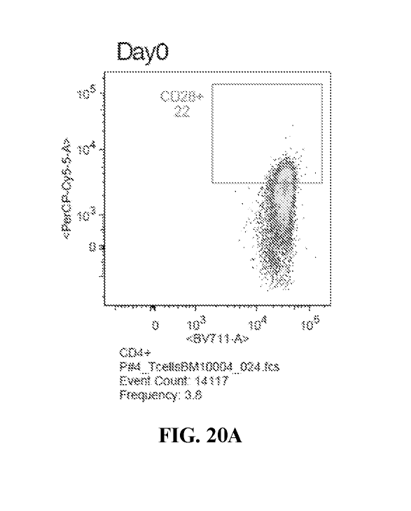

FIGS. 19A to 19C show gating strategy for counting CD8+/CD137+ cells after stimulation with Dynabeads® or aAPCs. Only beads group had increased 4-1BB expression by day 14.

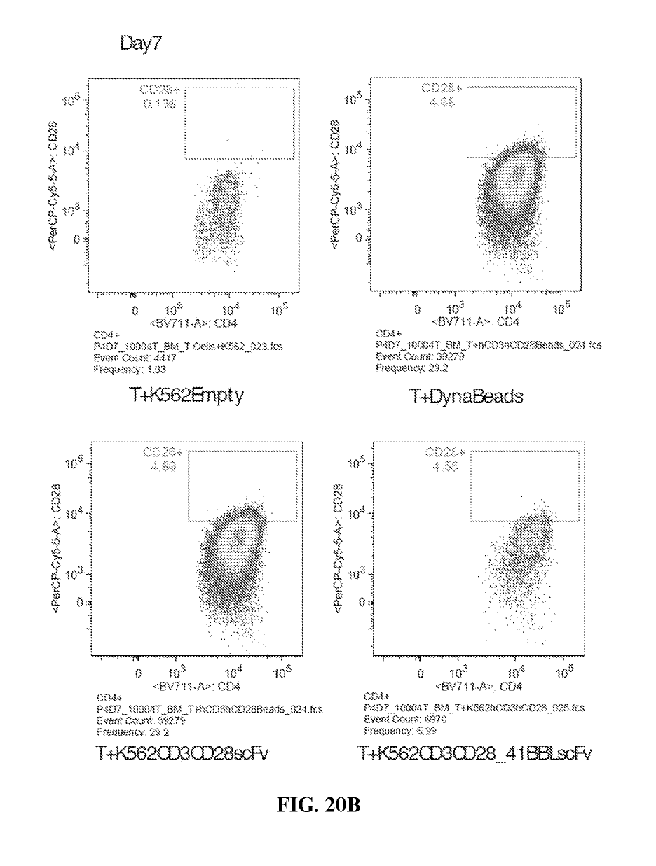

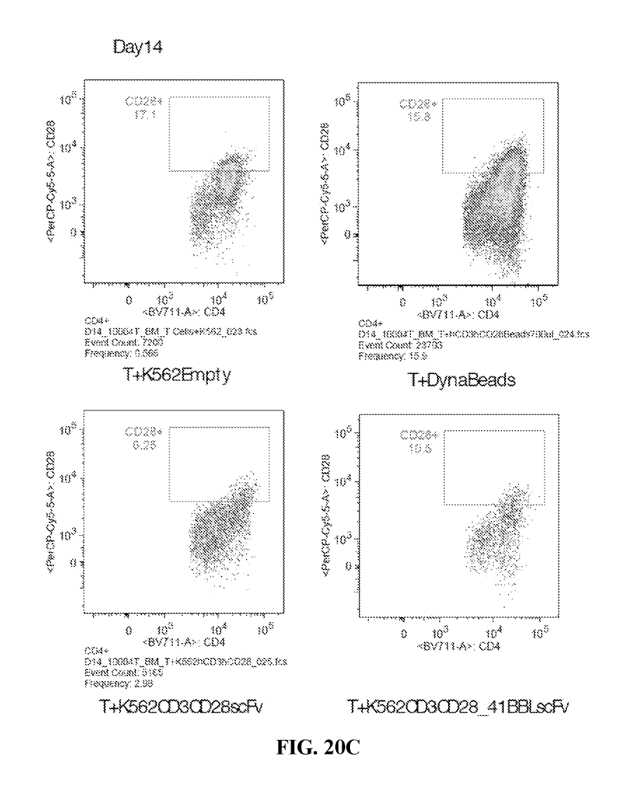

FIGS. 20A to 20C show gating strategy for counting CD4+/CD28+ cells after stimulation with Dynabeads® or aAPCs.

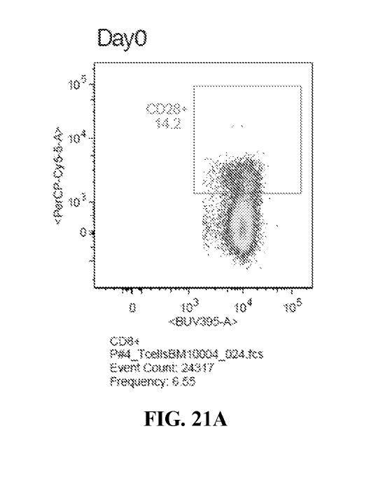

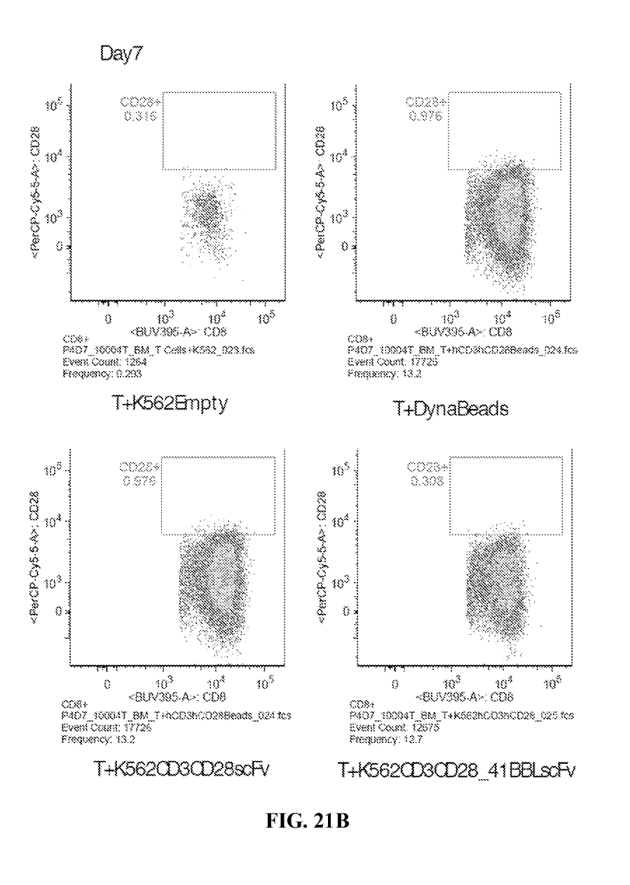

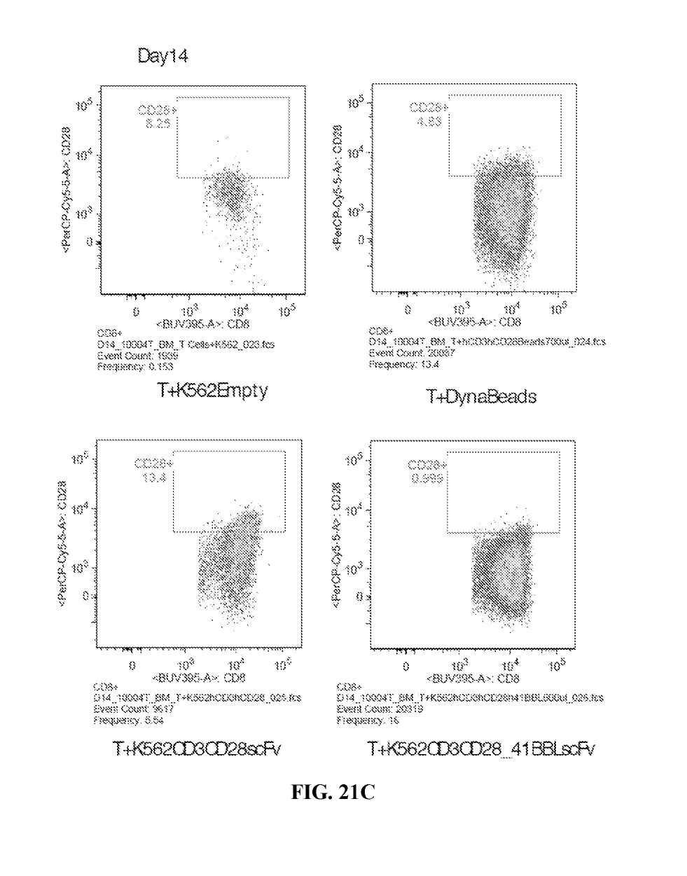

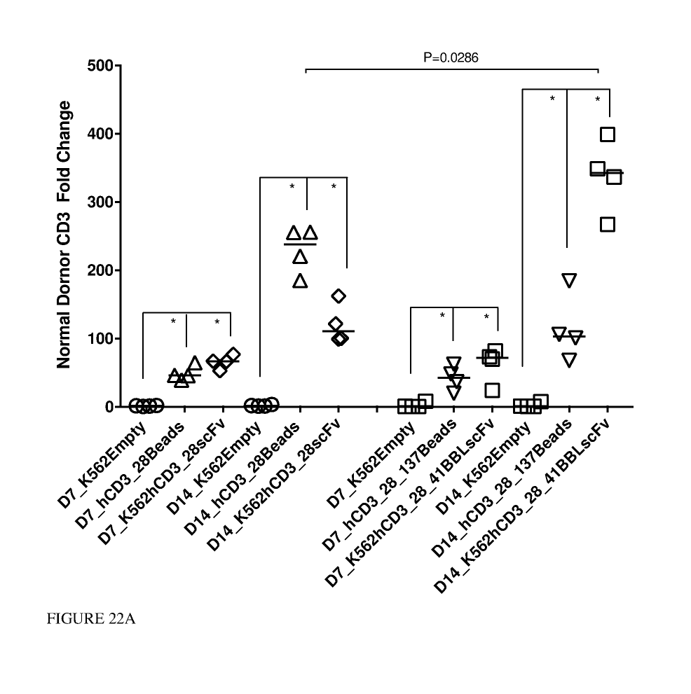

FIGS. 21A to 21C show gating strategy for counting CD8+/CD28+ cells after stimulation with Dynabeads® or aAPCs. This patient had relatively low CD28 expression initially, did not increase, but decreased on all groups.

### Example 2

### T-Cell Expansion

Normal Donor T Cell Expansion: 4 PBMCs from Stemcell Technologies were enriched with T cell isolation kit and T cells were expanded with hCD3CD28 double Beads, K562hCD3CD28scFv double APCs and K562 empty cells as a negative control; the other 4 PBMCs samples from AllCells were having T cell enrichment first, then T cells were expanded with hCD3CD28CD137triplesbeads, K562hCD3scFvCD28scFvCD137L triple APCs and K562 cells as a negative control. A 15 multicolor flow cytometry panel was performed on day0, day7 and day14. The Mann-Whitney and Wilcoxon test are used for statistical analysis.

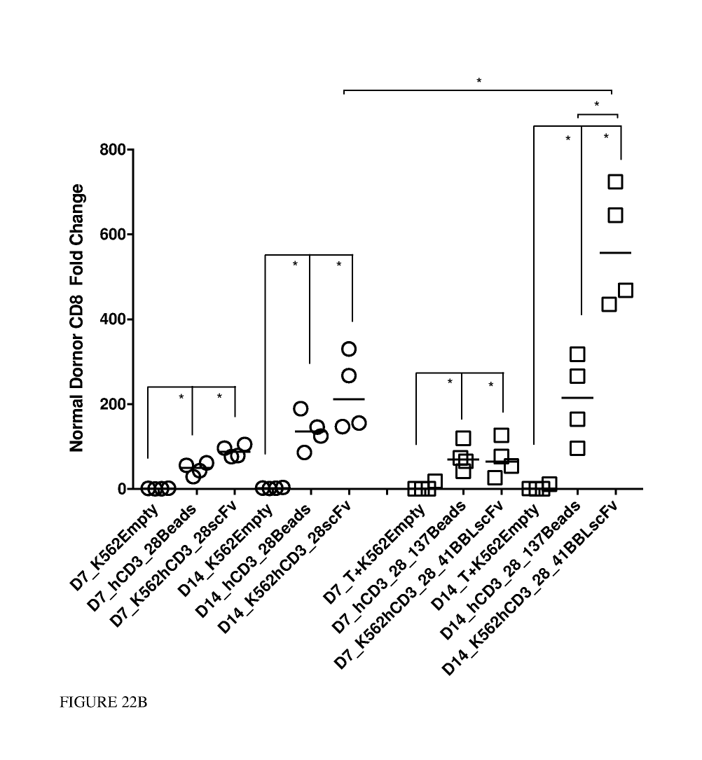

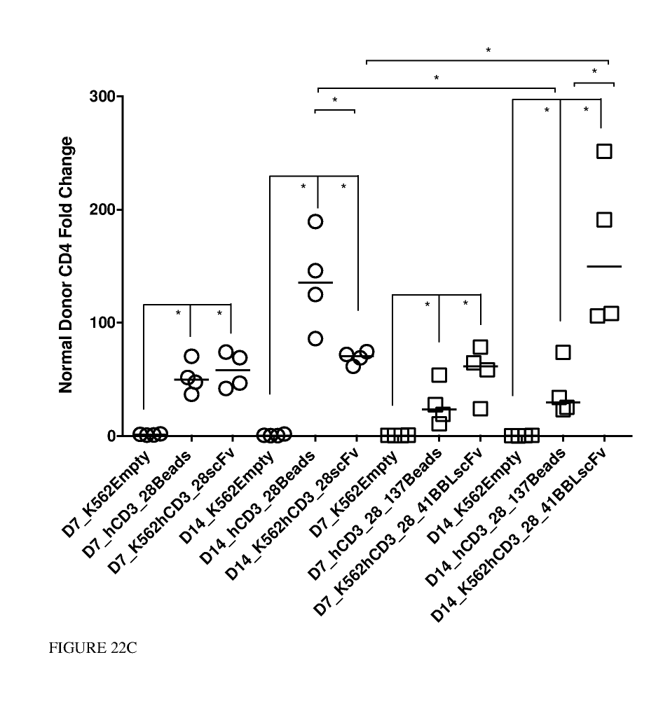

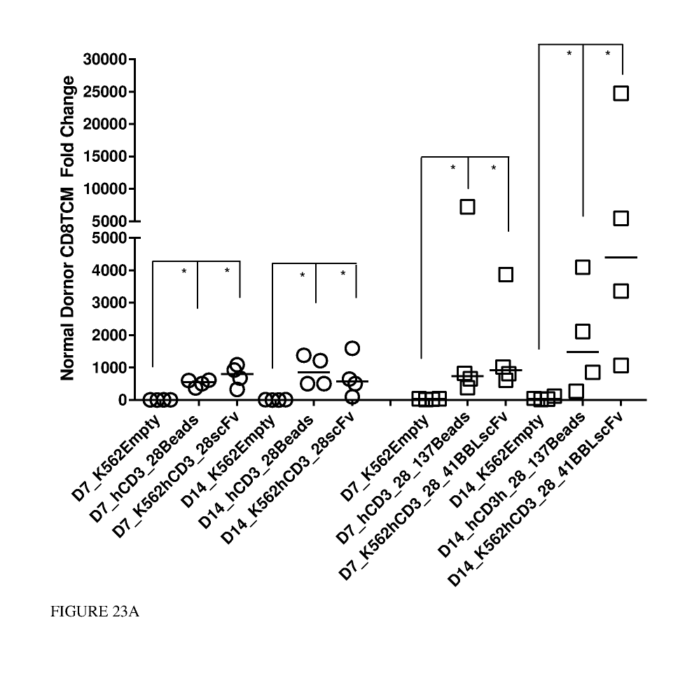

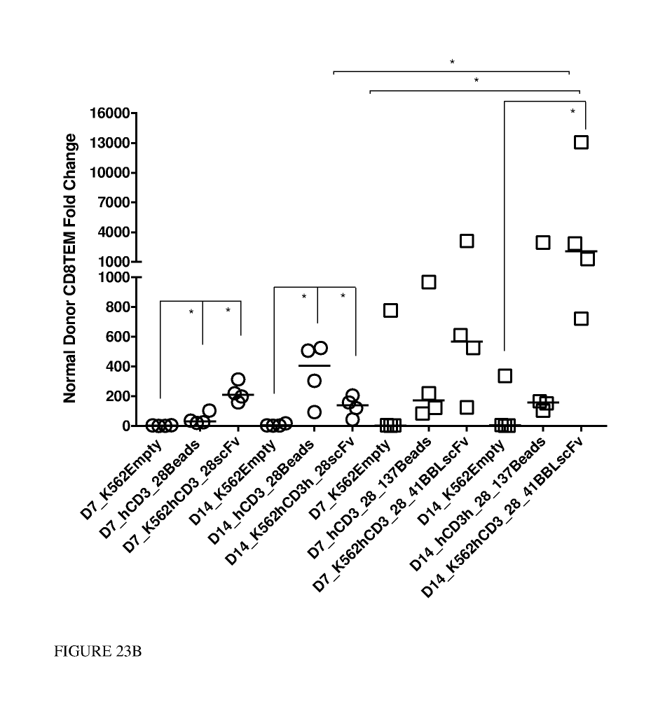

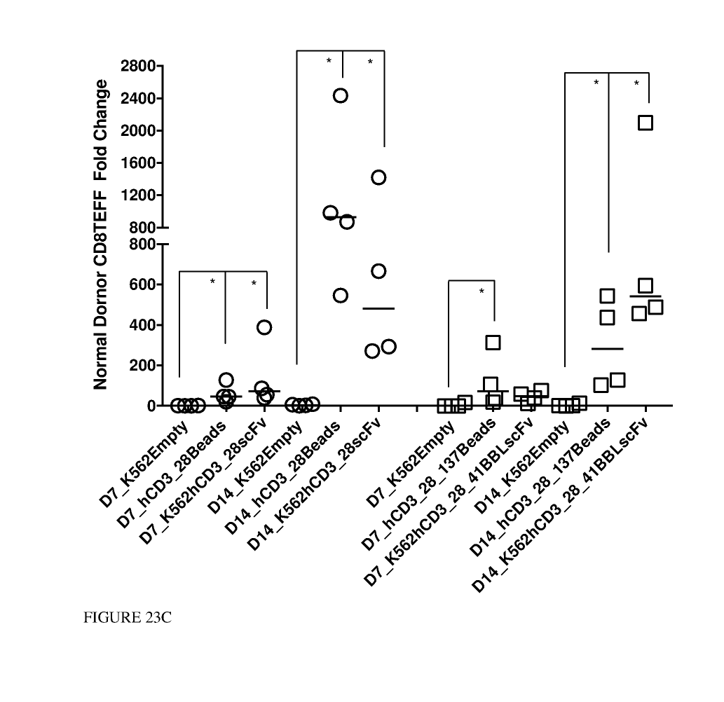

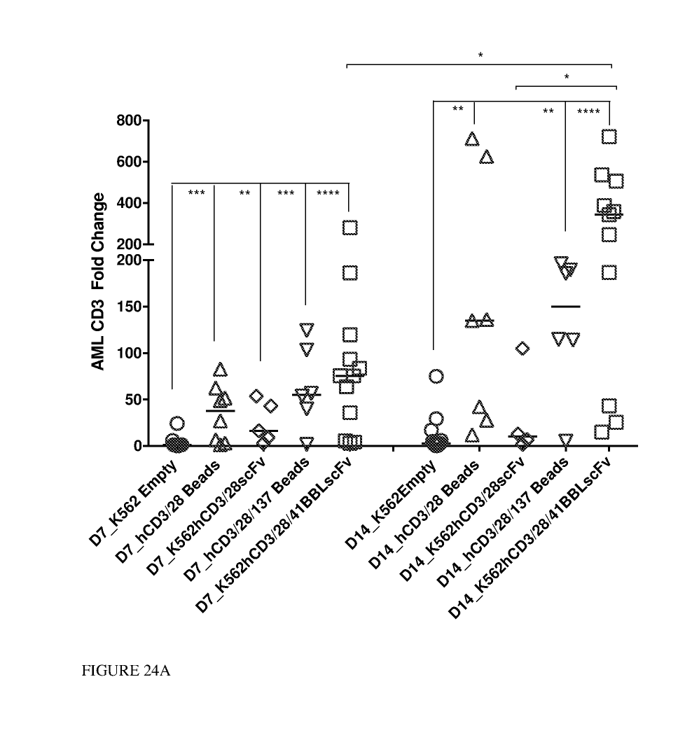

As can be seen in FIGS. 22A-C and 23A-C, K562hCD3scFvCD28scFvCD137L triple APCs shows significant CD3 T cell expansion (260-400 fold), significant CD8 expansion (430-720 fold) and good CD4 expansion (105-250 fold). Using double beads, CD3 expansion (180-250 fold) and CD4 expansion (80-180 fold) is also shown. CD8 did not have comparable expansion.

Triple APCs group expanded CD8 TCM (1,000-24,000 fold) and TEM (720-13,000) the most by day 14, and continues to expand both TCM and TEM from day 7 to day 14. The triple APCs group (450-2,100 fold) also shows CD8 TEFF increase as double beads group (540-2,400 fold) does.

AML T Cell Expansion:

K562 empty and K562hCD3scFvCD28scFvCD137L triple APCs group: 12 samples day 7 data collection, 11 samples day 14 data collection.

hCD3CD28 double Beads group: 8 samples day 7 data collection, 6 samples day14 data collection.

K562hCD3CD28scFv double APCs: 5 samples samples day 7 data collection, 4 samples day14 data collection. (Note: the groups above don't have the first sample data on day 14.)

hCD3scFvCD28scFvCD137L triple beads: 6 samples day 7 data collection, 6 samples day14 data collection.

All AML bone marrow samples had Ficoll isolation of mononuclear cells first, went through CD3+ T cell enrichment, then T cells were for day 0 flow phenotype and for T cell expansion in vitro for 14 days, then exact flow phenotype and analysis were performed on day 7 and day 14 as the same healthy donor had.

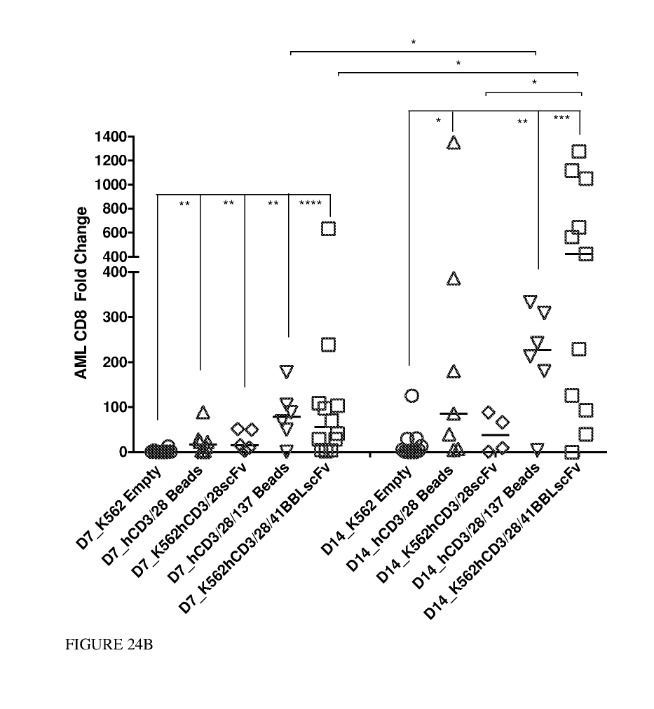

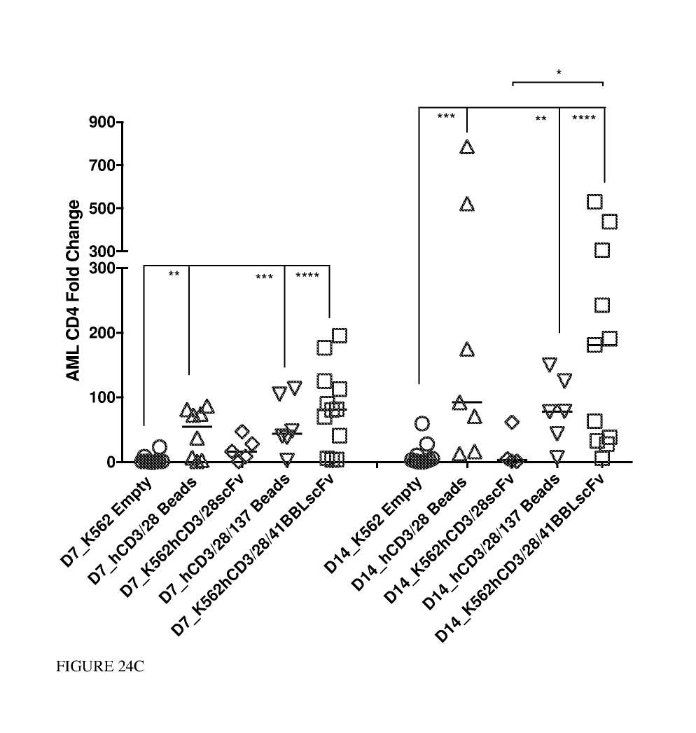

As can be seen in FIGS. 24A-C and 25A-C, 562hCD3scFvCD28scFvCD137L triple APCs still has the best CD3 T cell expansion, most of the patients T cells expanded from 180 to 720 fold, three samples showed low fold from 14 to 40 by day 14; most of the samples have the best CD8 expansion from 100 to 1,270 fold and decent CD4 expansion from 60 to 530 fold. Triple and double beads were the second best CD3 and CD8 expansion, double beads CD4 has nice expansion as triple APCs achieved.

Triple APCs showed significant expansion of CD8 TCM (100-13,000 fold) on day 7 and maintained the level till day 14 in AML patient bone marrow T cell expansion compared with the other groups, but some samples did not reach the same level as healthy donor T cell had. And most CD8 TEM in triple APCs demonstrated the best expansion from 500 to 15,000 fold by day 14, CD8 TEFF from this group also showed comparable increase from 100-1,400 fold as double beads had a decent CD4 expansion from 50 to 800 fold.

Melanoma TIL Expansion:

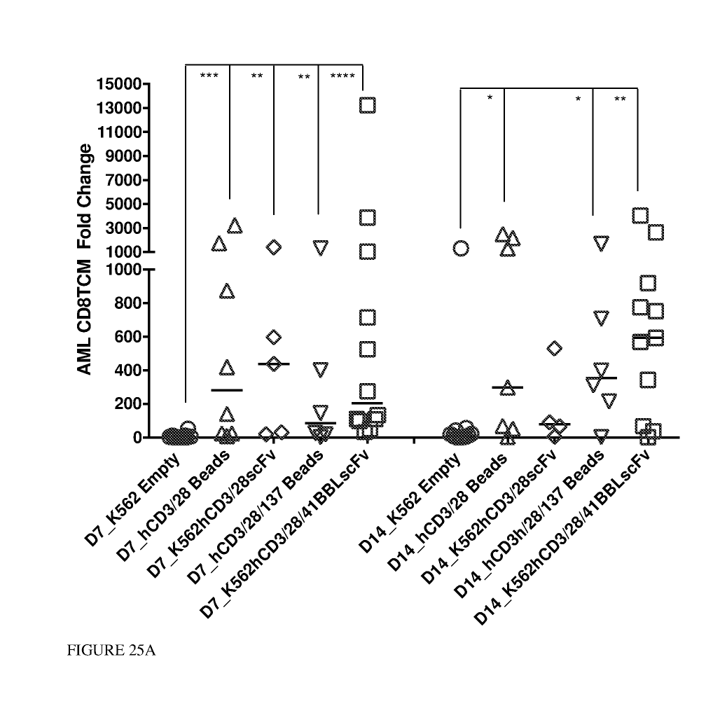

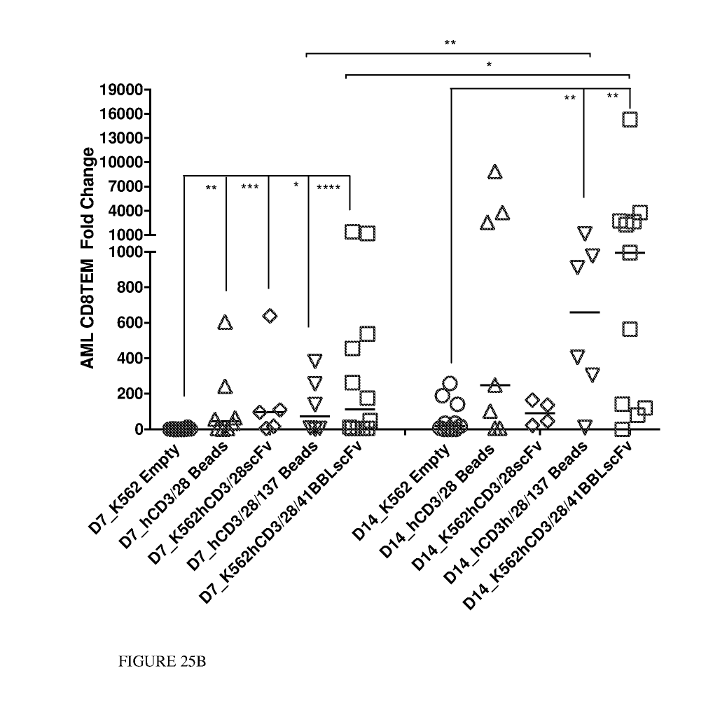

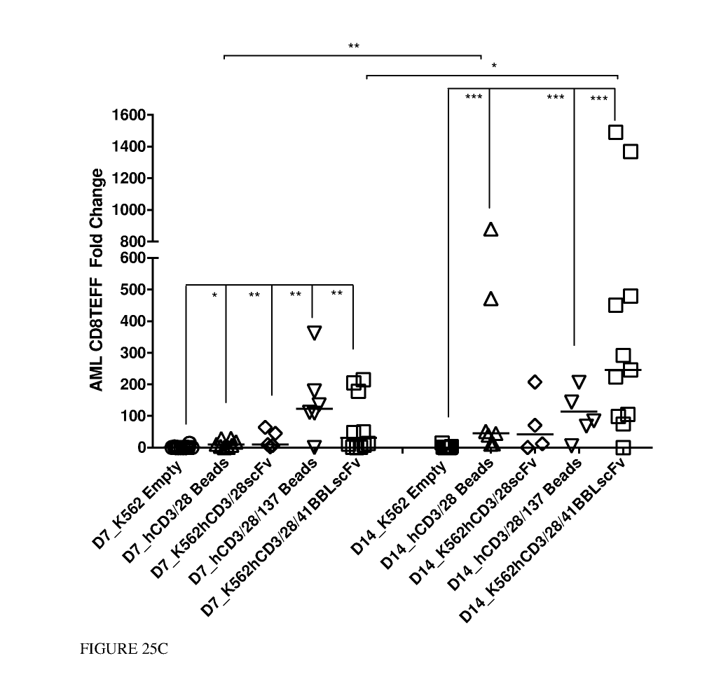

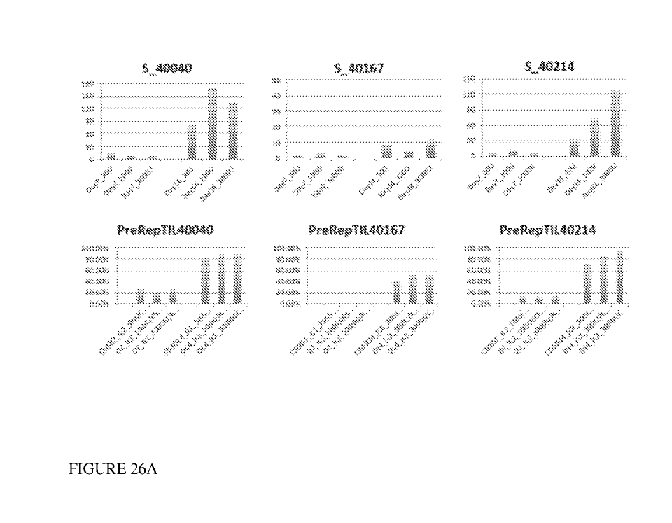

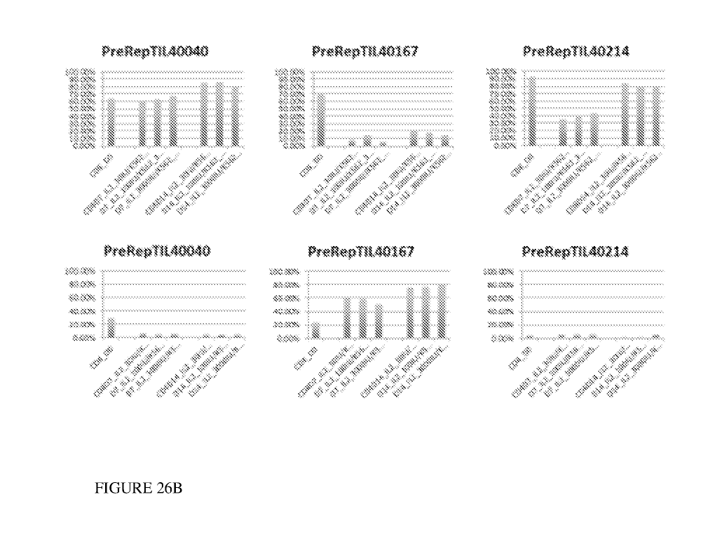

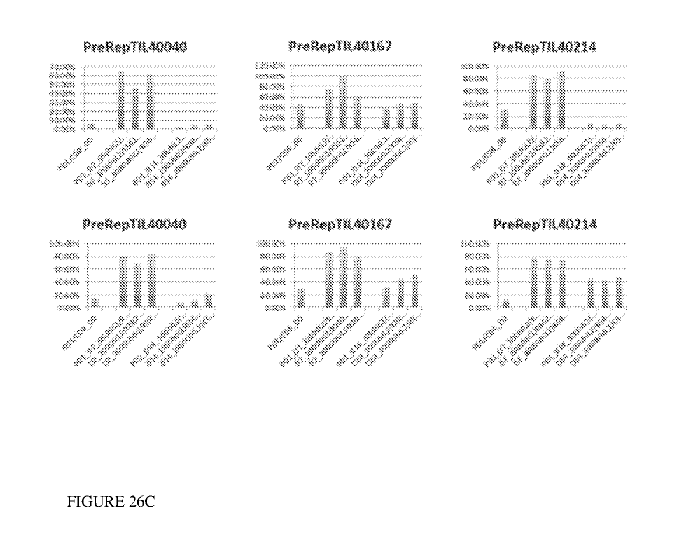

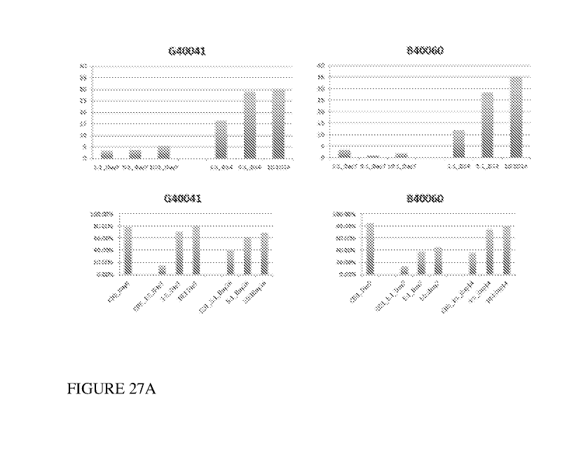

APCs K562hCD3scFvCD28scFvCD137L:TIL—1:1 ratio at 3000 U, 100 U, 30 U/ML of hIL2 concentration cultured with 3 different TIL samples. Looking into T cell fold change, CD3, CD4 and CD8% change, and the expression of TIM3, LAG3, PD1 and CD25 on both CD4 and CD8 T cells. Notes regarding FIGS. 25 and 26: aAPCs K562hCD3scFvCD28scFvCD137L titration with two TIL samples; TILs:aAPCs at 1:1, 5:1 and 10:1; hIL2-100 units/ml; and G40041 is a good TIL, B40060 is a bad TIL.

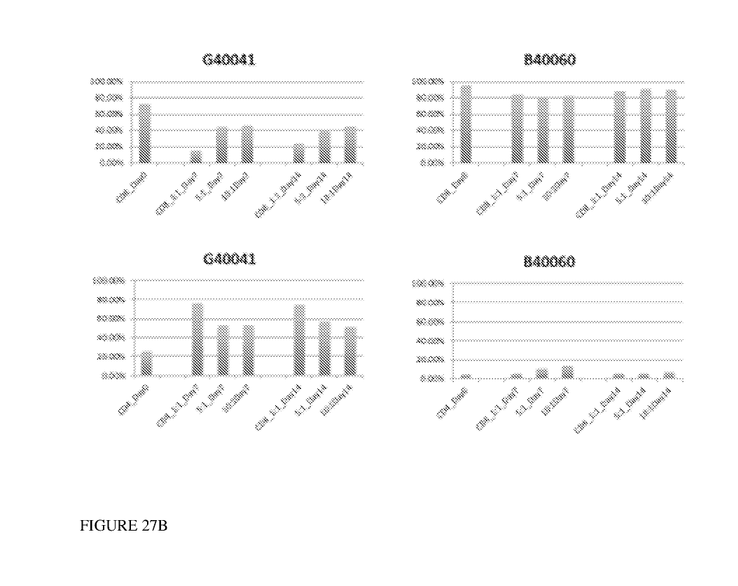

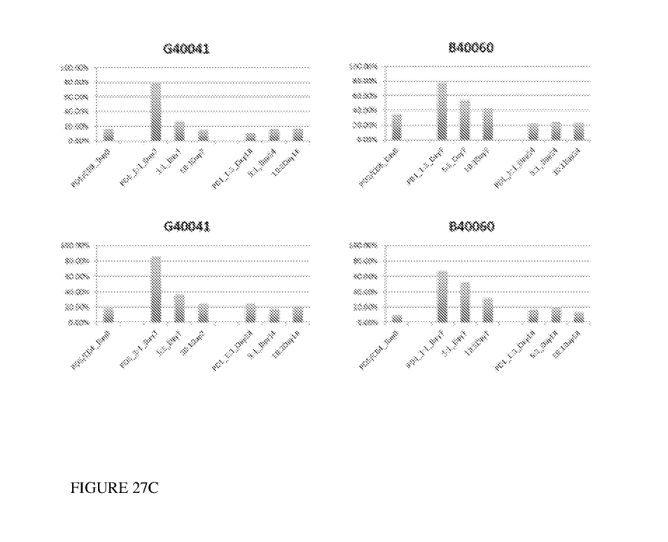

As can be seen in FIGS. 26 and 27, hIL2 dosage did not have a significant impact on melanoma TIL expansion. A lower dose of IL2 can help with T cell exhaustion. It can also be seen that the artificial APCs to TIL ratio does make TIL expand differently, so that the less APCs, the more TIL are expanded.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for expanding an immune cell isolated from a subject for autologous immune therapy, comprising
a) providing an artificial antigen presenting cell (aAPC) comprising a cell having a membrane,
wherein the cell secretes one or more single chain variable fragment (scFv) antibodies that bind a T cell inhibitory molecule, or a combination thereof,
wherein the cell contains on its membrane:
i) one or more scFv that selectively bind CD3,
ii) one or more scFv or ligands that bind a co-stimulatory molecule on T-cells; and
b) contacting the isolated immune cell with an effective amount of the aAPC to expand the immune cell in an amount effective for immunotherapy.

2. The method of claim 1, wherein the immune cell comprises a tumor infiltrating lymphocyte (TIL) or a marrow-infiltrating lymphocyte (MIL).

3. (canceled)

4. The method of claim 1, wherein the immune cell comprises a natural killer (NK) cell, an NK-T cell, a cytokine-induced memory NK cell, a cytokine-induced killer (CIK) cell, or a γδ T cell.

5. The method of claim 1, wherein the T cell inhibitory molecule comprises PD1, PDL1, or a combination thereof.

6. The method of claim 1, wherein the T cell inhibitory molecule comprises CTLA4, LAG3, TIM3, BTLA, CD160, 2B4, A2aR, KIR, or any combination thereof.

7. The method of claim 1, wherein the co-stimulatory molecule comprises CD28 and/or 4-1BB.

8. (canceled)

9. An immune cell produced by the method of claim 1.

10. A method for expanding tumor infiltrating lymphocytes for use in immunotherapy, comprising
a) providing an artificial antigen presenting cell (aAPC) comprising a cell having a membrane,
wherein the cell secretes one or more single chain variable fragments (scFv) that bind a T cell inhibitory molecule, or a combination thereof,
wherein the cell contains on its membrane:
i) one or more scFv that selectively bind CD3,
ii) one or more scFv or ligand that binds a co-stimulatory molecule on T-cells;
b) expanding tumor infiltrating lymphocytes (TILs) from a biopsy of a tumor from a subject;
c) screening the TILs for tumoricidal activity using flow cytometry to detect CD107 expression; and
d) contacting the tumoricidal TILs with an effective amount of the aAPC to expand the tumoricidal TILs.

11. The method of claim 10, further comprising infusing the expanded tumoricidal TILs into the subject in an effective amount to treat the tumor.

12. The method of claim 10, wherein the T cell inhibitory molecule comprises PD1, PDL1, or a combination thereof.

13. The method of claim 10, wherein the T cell inhibitory molecule comprises CTLA4, LAG3, TIM3, BTLA, CD160, 2B4, A2aR, KIR, or any combination thereof.

14. The method of claim 10, wherein the co-stimulatory molecule comprises CD28 and/or 4-1BB.

15. (canceled)

16. The method of claim 10, wherein the scFv antibody or ligand that binds the 4-1BB comprises a 4-1BBL.

17. The method of claim 10, wherein the co-stimulatory molecule comprises glucocorticoid-induced tumor necrosis factor-related receptor (GITR).

18. The method of claim 11, wherein the co-stimulatory molecule comprises CTLA4, PD-1, ICOS, CD25, TIM3, LAG3, PD1, CD40, CD137, OX40, CD2, LFA-1, CD28, CD154, BTLA, CD160, TIM 1, TIM 4, or any combination thereof.

19. The method of claim 11, wherein the cell further contains on its membrane an scFv antibody or ligand that selectively binds a cytokine receptor.

20. The method of claim 19, wherein the cytokine receptor comprises IL2R, IL7R, IL12R, IL15R, IL18R, IL10R, or any combination thereof.

21. The method of claim 11, wherein the cell line comprises a K562, NIH/3T3, Chinese hamster ovary (CHO), or Human Embryonic Kidney (HEK) cell line.

22. The method of claim 11, wherein the cell comprises contains on its membrane an scFv antibody that selectively bind CD3, an scFv that selectively binds CD28, and a 4-1BBL.

23. A tumor infiltrating lymphocyte produced by the method of claim 11.
